(19) United States
(12) Patent Application Publication
(10) Pub. No.: US 2007/0123718 A1
(43) Pub. Date: May 31, 2007

(54) Novel 4-amino-2(5H)-furanones

(75) Inventor: Eric Lattmann, Birmingham (GB)

Correspondence Address: SYNNESTVEDT LECHNER & WOODBRIDGE LLP, P O BOX 592, 112 NASSAU STREET, PRINCETON, NJ, 08542-0592, US

(73) Assignee: Aston University, Birmingham (GB)

(21) Appl. No.: 10/558,550

(22) PCT Filed: May 27, 2004
(86) PCT No.: PCT/GB04/02256

§ 371 Date: Nov. 2, 2006

(30) Foreign Application Priority Data
May 30, 2003 (GB) 0312360.1

(51) Int. Cl. C07D 307/62; A61K 31/365

(52) U.S. Cl. 549/315; 514/471

(57) ABSTRACT

The present invention relates to compounds of formula (1): wherein X is selected from hydrogen, a halogen, a substituted or unsubstituted cyclic and heterocyclic moiety, substituted or unsubstituted, linear or branched alkyl, alkyloxy, alkylcarbonyl, alkyloxycarbonyl, alkenyl, alkenyloxy, alkenylcarbonyl, alkenyloxycarbonyl, alkynyl, alkynyloxy, alkynylcarbonyl, alkynyloxycarbonyl, aryl, benzyl, arlyoxy, arylcarbonyl, aryloxycarbonyl and sulphur equivalents of said oxy, carbonyl and oxycarbonyl moieties, R is selected from hydrogen, a halogen, an amide, a substituted or unsubstituted cyclic and heterocyclic moiety, substituted or unsubstituted, linear or branched alkyl, alkyloxy, alkylcarbonyl, alkyloxycarbonyl, alkenyl, alkenyloxy, alkenylcarbonyl, alkenyloxycarbonyl, alkynyl, alkynyloxy, alkynylcarbonyl, alkynyloxycarbonyl, aryl, benzyl, arlyoxy, arylcarbonyl, aryloxycarbonyl and sulphur equivalents of said oxy, carbonyl and oxycarbonyl moieties, and R¹ and R² arc each independently selected from H, C₁₋₁₈ straight, branched or cyclic, saturated, unsaturated and aromatic hydrocarbyl groups, which aromatic groups may be heterocyclic, cyclic or acyclic and which may optionally be substituted by alkyl, alkoxy, or halo; or R¹ and R², when taken together with the N-atom to which they are bonded, may form an N-containing saturated, unsaturated or partially unsaturated ring system comprising 3 to 10 ring atoms selected from C, N and O, optionally substituted at any position of the ring by a substituent selected from a halogen, a substituted or unsubstituted cyclic and heterocyclic moiety, substituted or unsubstituted, linear or branched alkyl, alkyloxy, alkylcarbonyl, alkyloxycarbonyl, alkenyl, alkenyloxy, alkenylcarbonyl, alkenyloxycarbonyl, alkynyl, alkynyloxy, alkynylcarbonyl, alkynyloxycarbonyl, aryl, benzyl, arlyoxy, arylcarbonyl, aryloxycarbonyl, sulphur equivalents of said oxy, carbonyl and oxycarbonyl moieties, and oxo. The invention also relates to their uses as CCK receptor ligands and CCK antagonists.

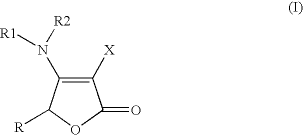

The present invention relates to novel 4-amino-2(5H)-furanones, their preparation and their use as non-peptide CCK ligands, particularly in pharmaceutical formulations thereof.

Cholecystokinins (CCKs) act as anti-opioid peptides. CCK was initially described as a regulatory hormone found in endocrine cells of the gastro-intestinal (GI) tract. Some CCKs share a common amino acid sequence with gastrin, which is involved in control of C gastric acid and pepsin secretion. CCKs have also been found throughout the central nervous system (CNS), where they are believed to act as a neurotransmitter and/or modulator of many important functions. There are various known structures of CCK, identified with reference to the number of amino acids they comprise. For example, CCK-8 is a naturally-occurring predominating CCK peptide and, having only eight amino acids, is the minimum fully-active sequence, although small amounts of CCK-4 may also be present.

CCK plays an.important role in the invasiveness and the production of matrix metalloproteinase-9 (MMP-9) in human pancreatic cancer cell lines. The pathway of the invasiveness may be associated with MM?-9 of those lines regulated by CCK.

The gut hormone cholecystokinin exerts various actions on the gastrointestinal tract, including the regulation of growth. The hormone has been reported to induce hypertrophy and hyperplasia of the pancreas and to enhance chemically-induced pancreatic carcinogenesis in animals. Stimulation of endogenous cholecystokinin secretion through the induction of deficiency of intraintestinal proteases and bile salts by trypsin-inhibiting nutrients, bile salt-binding drugs or surgical intervention is also capable of stimulating growth and tumour development in the rat. In man, factors suggested to increase the risk of pancreatic cancer, such as a high-fat and high-protein diet or gastrectomy, are known to stimulate plasma cholecystokinin secretion. Receptors for cholecystokinin have been demonstrated on human pancreatic adenocarcinomas, and cholecystokinin has been demonstrated to enhance the growth of xenografted pancreatic cancer and to inhibit growth of gastric and bile duct cancer.

There are two subtypes of CCK receptor which were initially termed as type-A and type-B, reflecting their preferential localisation in the alimentary tract and in the brain, respectively. Recently, these receptors have been re-named as CCK1 and CCK2, respectively, although the original designation is used herein below with respect to the present invention. The molecular cloning of two CCK receptor subtypes, one from rat and human pancreas and one from human brain, has confirmed the pharmacological classification of CCK receptors. Both CCK1 and CCK2 receptors belong to the family of G-protein coupled receptors. However, the differential distribution of CCK1 and CCK2 receptors in the peripheral vs. central nervous system is not absolute, and CCK1 receptors have been shown to be present in discrete regions of the CNS, including the spinal cord, particularly in primates.

The functions of the CCK1 receptors in the brain is poorly understood, whereas the CCK2 receptor is known to mediate anxiety, panic attacks, satiety and pain. Therefore, antagonists to CCK and to gastrin have been useful for preventing and treating CCK-related and/or gastrin-related disorders of the GI and CNS of animals, especially of humans. Just as there is some overlap in the biological activities of CCK and gastrin, antagonists also tend to have affinity for both receptors. In a practical sense, however, there is enough selectivity for the respective receptors that greater activity against specific CCK- or gastrin-related disorders can often also be identified.

Selective CCK antagonists are themselves useful in treating CCK-related disorders of the appetite regulatory systems of animals as well as in potentiating and prolonging opiate-mediated analgesia, thus having utility in the treatment of pain, while selective gastrin antagonists are useful in the modulation of CNS behaviour, as a palliative for gastrointestinal neoplasms, and in the treatment and prevention of gastrin-related disorders of the GI system in humans and animals, such as peptic ulcers, Zollinger-Ellison syndrome, antral G cell hyperplasia and other conditions in which reduced gastrin activity is of therapeutic value. Also, since CCK and gastrin also have trophic effects on certain tumours, antagonists of CCK and gastrin are useful in treating these tumours.,

Various chemical classes of CCK-receptor antagonists have been reported. These include pyrazolidinones showing good selectivity for CCKB receptors (Howbert, J. J. et. al.; Diphenylpyrazolidinone and benzodiazepine cholecystok-inin antagonists: A case of convergent evolution in medicinal chemistry., Bioorg. Med. Chem. Lett. 1993, 3, 875-880.), ureidoacetamides which are potent and selective ligands for CCK_{B}/gastrin receptors (WO 91/113874), ureidophenoxyacetanilides (Takeda, Y.et. al.; Synthesis of phenoxyacetic acid derivatives as highly potent antagonists of gastrin/ cholecystokinin-B receptors, Chem. Pharm. Bull. 1998, 46, 951-961), ureidomethylcarbamoylphenylketones (Hagishita, S.; et. al., Ureido-methylcarbamoyl-phenylketones as selective CCK_{B} receptor antagonists. Bioorg. Med. Chem. 1997, 5, 1695-1714), and ureidobenzodiazepine derivatives (Evans, B. E.; et. al., Design of potent, orally effective, non peptidal antagonists of the peptide hormone cholecystokinin, Proc. Natl. Acad. Sci. USA 1986, 83, 4918-4922).

It is an object of the presentinvention to provide novel 4-amrino-2(5H)-furanone derivatives, which preferably act as CCK ligands, and pharmaceutical formulations thereof

According to the present invention there is provided a compound of formula (I): 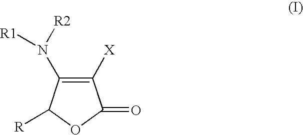
wherein

X is selected from hydrogen, a halogen, a substituted or unsubstituted cyclic and heterocyclic moiety, substituted or unsubstituted, linear or branched alkyl, alkyloxy, alkylcarbonyl, alkyloxycarbonyl, alkenyl, alkenyloxy, alkenylcarbonyl, alkenyloxycatbonyl, alkynyl, alkynyloxy, alkynylcarbonyl, alkynyloxycarbonyl, aryl, benzyl, arlyoxy, arylcarbonyl, aryloxycarbonyl and sulphur equivalents of said oxy, carbonyl and oxycarbonyl moieties,

R is selected from hydrogen, a halogen, an amide, a substituted or unsubstituted cyclic and heterocyclic moiety, substituted or unsubstituted, linear or branched alkyl, alkyloxy, alkylcarbonyl, alkyloxycarbonyl, alkenyl, alkenyloxy, alkenylcarbonyl, alkenyloxycarbonyl, alkynyl, alkynyloxy, alkynylcarbonyl, alkynyloxycarbonyl, aryl, benzyl, arlyoxy, arylcarbonyl, aryloxycarbonyl and sulphur equivalents of said oxy, carbonyl and oxycarbonyl moieties, and

R¹ and R² are each independently selected from H, C₁₋₁₈ straight, branched or cyclic, saturated, unsaturated and aromatic hydrocarbyl groups, which aromatic groups may be heterocyclic, cyclic or acyclic and which may optionally be substituted by alkyl, alkoxy, orshalo; or R¹ and R², when taken together with the N-atom to which they are bonded, may form an N-containing saturated, unsaturated or partially unsaturated ring system comprising 3 to 10 ring atoms selected from C, N and O, optionally substituted at any position of the ring by a substituent selected from a halogen, a substituted or unsubstituted cyclic and heterocyclic moiety, substituted or unsubstituted, linear or branched alkyl, alkyloxy, alkylcarbonyl, alkyloxycarbonyl, alkenyl, alkenyloxy, alkenylcarbonyl, alkenyloxycarbonyl, alkynyl, alkynyloxy, alkynylcarbonyl, alkynyloxycarbonyl, aryl, benzyl, arlyoxy, arylcarbonyl, aryloxycarbonyl, sulphur equivalents of said oxy, carbonyl and oxycarbonyl moieties, and oxo.

Preferably said alkyl-containing moieties (e.g. alkyl, alkyloxy etc.) are C₁-C₁₈, more preferably C₁-C₁₂ and most preferably C₁ to C₆.

Preferably said alk-enyl- and said alkynyl-containing moieties are C₂-C₁₈, more preferably C₂-C₁₂ and most preferably C₂ to C₆.

Preferred substituents on a ring system formed by R¹ and R² are C₁₋₆ alkyl or alkoxy, phenyl, benzyl, phenyl (C₂₋₄) alkenyl, phenoxy, benzyloxy, halo, oxo or alkyloxycarbonyl.

Especially preferred groups for R¹ and R² are, independently, H, C₁₋₆ alkyl (eg. hexyl and i-propyl), alkenyl, and alkynyl, benzyl, and cyclohexyl.

In a first series of embodiments, one of R¹ and R² is H, C₁₋₆ alkyl or benzyl and the other is C₁₋₆ alkyl, phenyl, benzyl, phenyl (C₂₋₄) alkyl, especially phenethyl, cyclohexyl, 1,3-dihydro-3H-pyrazolyl or morpholin4-yl.

When R¹ and R² form a secondary amine (non-cyclic-formi), these exhibit two isomeric forms, both of which are encompassed by formula (I), as are all other isomers, when applicable to a given structural formula.

In a second series of embodiments, R¹ and R² taken together with the N-atom to which they are bonded, form optionally-substituted: pyrrolidinyl, piperidinyl, benzimidazolyl, pyrrolyl, pyrazolyl, tetrahydropyrazinyl, dihydropyrazolyl, pyrazolyl, 2,3-dihydro-IH-indol-1-yl, pipetrazin-1-yl, morpholin-4-yl or pyrid-1-yl.

Suitable substituents on the heterocyclic ring of the second series of embodiments are methyl, benzyl, phenyl, alkoxycarbonyl and oxo. Preferably, said heterocyclic ring is mono- or di-substituted.

Preferably, X is H, halo (F, Br, Cl, I) or methyl, and is most preferably chloro.

Preferably, R is H, halo, imidazolidinoyl, alkoxy, alkenoxy, alk-ynyloxy, alkylcarbonyloxy, alkylcarbonylmethyl, hydrazonoalkylmethyl, R³—N(R⁴CO)—, R³—N(R⁴)—CO—O—, and R³—N(R⁴)—O—, wherein R³ and R⁴ are independently selected from C₁₋₁₈ straight, branched or cyclic, saturated, unsaturated and aromatic hydrocarbyl groups, which aromatic groups may optionally be substituted by CIl alkyl or alkoxy, and halo.

R³ is preferably a C₆₋₁₀ aromatic group, especially optionally substituted phenyl, naphthyl or benzyl; particularly where the phenyl group is optionally substituted by methyl, methoxy or chloro.

R⁴ is preferably H, methyl or ethyl

More preferably, R is methoxy, ethenyloxy, propyn-2-yloxy, methylcarbonyloxy or optionally substituted phenyl.

It will be understood that formula (I) is intended to embrace all possible isomers, including optical isomers and mixtures thereof, including racemates. In addition, the present invention includes within its scope prodrugs of the compounds of formula (I). In general, such prodrugs will be functional derivatives of the compounds of formula (I) which are readily convertible ini vivo into the required compound of formula (I). Conventional procedures for the selection and preparation of suitable prodrug derivatives are described, for example, in “Design of Prodrugs”, ed H. Bungaard, Elsevier, 1985.

The scope of the invention also extends to salts, particularly physiologically acceptable salts and hydrates of the compounds of formula (I).

The pharmaceutically acceptable salts of the compounds of formula (I) include the conventional non-toxic salts or the quarternary ammonium salts of the compounds of formula (I) formed, eg, from non-toxic inorganic or organic acids. The pharmaceutically acceptable salts of formula (I) also include those formed from a base, such as an alkali or alkaline earth metal hydroxide, or an organic base, such as an amine or a quarternary ammonium hydroxide.

Highly preferred compounds in accordance with the invention are:—

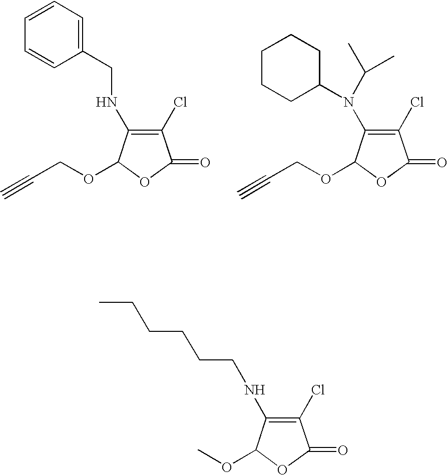

The present invention also resides in the use of a compound of the first aspect as a CCK receptor ligand and/or as a CCK antagonist. Preferably, said use is as a selective CCK1 or CCK2 ligand.

The ability of the compounds of formula (I) to antagonise CCK by acting as CCK-receptor ligands makes these compounds useful as pharmacological agents for mammals, especially humans, for the treatment and prevention of disorders wherein CCK and/or gastrin may be involved.

Therefore the present invention in a second aspect resides in a method of treatment of a mammal afflicted with a CCK-related condition, or prophylaxis in a mammal at risk of a CCK-related condition by administration of a therapeutically effective amount of a compound of the first aspect of the invention.

The invention also resides in a pharmaceutical formulation comprising a compound of said first aspect in admixture with a pharmaceutically acceptable carrier therefor.

The invention further resides in the use of a compound of the first aspect in the preparation of a medicament, particularly a medicament for the treatment or prophylaxis of a CCK-related disorder.

Examples of CCK-related conditions include GI disorders, especially such as irritable bowel syndrome, gastro-oesophageal reflux disease or ulcers, excess pancreatic or gastric secretion, acute pancreitis, or motility disorders; CNS disorders caused by CCK interactions with dopamine, such as neuroleptic disorders, tardive dyskinesia, Parkinson's disease, psychosis or Gilles de la Tourette syndrome; disorders of appetite regulatory systems; Zollinger-Ellison syndrome; antral G cell hyperplasia; or pain (potentiation of opiate analgesia).

The treatment of opiate-resistant severe clinical pain may represent the most important of the CNS applications, but other applications based on the interaction between CCK and dopamine in forebrain could also deserve clinical exploration

The comnpounds of the invention may further be useful in the treatment or prevention of additional central nervous system disorders including neurological and psychiatric disorders. Example of such central nervous system disorders include anxiety disorders and panic disorders, wherein CCK is involved. Additional examples of central nervous system disorders include panic syndrome, anticipatory anxiety, phobic anxiety, panic anxiety, chronic anxiety and endogeneous anxiety.

The compounds of of the invention may further be useful in the treatment of oncologic disorders wherein CCK may be involved. Examples of such oncologic disorders include small cell adenocarcinomas and primary tumours of the central nervous system glial and neuronal cells. Example of such adenocarcinomas and tumours include, but are not C limited to, tumours of the lower oesophagus, stomach, intestine, colon and lung, including small cell lung carcinoma.

The compounds of the invention may further be used to control pupil constriction in the eye. The compounds may be used for therapeutic purposes during eye examinations and intra-ocular surgery in order to prevent miosis. They may further be used to inhibit miosis occurring in association with iritis, uveitis and trauma.

The compounds of the invention may further be useful. for preventing or treating the withdrawal response produced by chronic treatment or abuse of drugs or alcohol. Such drugs include, but are not limited to, cocaine, alcohol or nicotine.

The compounds of the invention may also be useful as neuroprotective agents, for example, in the treatment and/or prevention of neuro-degenerative disorders arising as consequence of such pathological conditions as stroke, hypoglycaemia, cerebral palsy, transient cerebral ischaemic attack, cerebral ischaenba during cardiac-pulmonary surgery or cardiac arrest, perinatal asphyxia, epilepsy, Huntingdon's chorea, Alzheimer's disease, amyotrophic lateral sclerosis, Parkinson's disease, olivo-pontocerebellar atrophy, anoxia such as from drowning, spinal cord and head injury, and poisoning by neurotoxins, including environmental neurotoxins.

The dosage administered to a patient will normally be determined by the prescribing physician and will generally vary according to the age, weight and response of the individual patient, as well as the severity of the patient's symptoms. However, in most instances, an effective therapeutic daily dosage will be in the range of from about 0.05 mg/kog to about 50 mg/kg of body weight and, preferably, of from 0.5 mg/kg to about 20 mg/kg of body weight administered in single or divided doses. In some cases, however, it may be necessary to use dosages outside these limits.

In the treatment of irritable bowel syndrome, for instance, 0.1 to 10 mg/kg of a CCK antagonist might be administered orally (p.o.), divided into two doses per day (b.i.d.). In treating delayed gastric emptying, the dosage range would probably be the same, although the drug might be administered either intravenously (i.v.) or orally, with the i.v. dose probably tending to be slightly lower due to a better availability. Acute pancreitis might be treated preferentially in an i.v. form, whereas spasm and/or reflex oesophageal, chronic pancreitis, post-vagotomy diarrhoea, anorexia or pain associated with biliary dyskinesia might indicate a p.o. form of administration.

In the effective treatment of panic syndrome, panic disorder, anxiety disorder and the like, preferably about 0.05 mg/kg to about 1.0 mg/kg of CCK antagonist may be administered orally (p.o.), in single or divided doses per day (b.i.d.). Other routes of administration are also suitable.

For directly introducing analgesia, anaesthesia or loss of pain sensation, the effective dosage range is preferably from about 100 mg/kg to about 1 mg/kg by intraperitoneal administration. Oral administration is an alternative route, as well as others.

While it is possible for an active ingredient to be administered alone as the raw chemical, it is preferable to present it as a pharmaceutical formulation. The formulations, both for veterinary and for human medical use, of the present invention comprise an active ingredient in association with a pharmaceutically acceptable carrier therefor and optionally other therapeutic ingredient(s). The carrier(s) must be ‘acceptable’ in the sense of being compatible with the other ingredients of the formulation and not deleterious to the recipient thereof.

Conveniently, unit doses of a formulation contain between 0.1 mg and 1 g of the active ingredient. Preferably, the formulation is suitable for administration from one to six, such as two to four, times per day. For topical administration, the active ingredient preferably comprises from 1% to 2% by weight of the formulation but the active ingredient may comprise as much as 10% w/w. Formulations suitable for nasal or buccal administration, such as the self-propelling powder-dispensing formulations described hereinafter, may comprise 0.1 to 20% w/w, for example about 2% w/w of active ingredient.

The formulations include those in a form suitable for oral, ophthalmic, rectal, parenteral (including subcutaneous, vaginal, intraperitoneal, intramuscular and intravenous), intra-articular, topical, nasal or buccal administration.

Formulations of the present invention suitable for oral administration may be in the form of discrete units such as capsules, cachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. The active ingredient may also be in the form of a bolus, electuary or paste. For such formulations, a range of dilutions of the active ingredient in the vehicle is suitable, such as from 1% to 99%, preferably 5% to 50% and more preferably 10% to 25% dilution. Depending upon the level of dilution, the formulation will be either a liquid at room temperature (in the region of about 20° C.) or a low-melting solid.

Formulations for rectal administration may be in the form of a suppository incorporating the active ingredient and a carrier such as cocoa butter, or in the form of an enema.

Formulations suitable for parenteral administration comprise a solution, suspension or emulsion, as described above, conveniently a sterile aqueous preparation of the active ingredient that is preferably isotonic with the blood of the recipient.

Formulations suitable for intra-articular administration may be in the form of a sterile aqueous preparation of the active ingredient, which may be in a microcrystalline form, for example, in the form of an aqueous microcrystalline suspension or as a micellar dispersion or suspension. Liposomal formulations or biodegradable polymer systems may also be used to present the active ingredient particularly for both intra-articular and ophthalmic administration.

Formulations suitable for topical administration include liquid or semi-liquid preparations such as liniments, lotions or applications; oil-in-water or water-in-oil emulsions such as creams, ointments or pastes; or solutions or suspensions such as drops. For example, for ophthalmic administration, the active ingredient may be presented in the form of aqueous eye drops, as for example, a 0.1-1.0% solution.

Drops according to the present inventioni may comprise sterile aqueous or oily solutions. Preservatives, bactericidal and fungicidal agents suitable for inclusion in the drops are phenylmercuric salts (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the eye. An eye lotion may comprise a sterile aqueous solution optionally containing a bactericide or preservative prepared by methods simirlar to those for the preparation of drops. Lotions or liniments for application to the slin may also include an agent to hasten drying and to cool the skin, such as an alcohol, or a softener or moisturiser such as glycerol or an oil such as castor oil or arachis oil.

Creams, ointments or pastes according to the present invention are semi-solid formulations of the active ingredient in a base for external application. The base may comprise one or more of a hard, soft or liquid paraffin, glycerol, beeswax, a metallic soap; a mucilage; an oil such as a vegetable oil, eg almond, corn, arachis, castor or olive oil; wool fat or its derivatives; or a fatty acid ester of a fatty acid together with an alcohol such as propylene glycol or macrogols. The formulation may also comprise a suitable surface-active agent, such as an anionic, cationic or non-ionic surfactant such as a glycol or polyoxyethylene derivatives thereof. Suspending agents such as natural gums may be incorporated, optionally with other inorganic materials, such as silicaceous silicas, and other ingredients such as lanolin.

Formulations suitable for administration to the nose or buccal cavity include those suitable for inhalation or insufflation, and include powder, self-propelling and spray formulations such as aerosols and atomisers. The formulations, when dispersed, preferably have a particle size in the range of 10 to 200μ.

Such formulations may be in the form of a finely commuted powder for pulmonary administration from a powder inhalation device or self-propelling powder-dispensing formulations, where the active ingredient, as a finely comminuted powder, may comprise up to 99.9% w/w of the formulation.

Self-propelling powder-dispensing formulations preferably comprise dispersed particles of solid active ingredient, and a liquid propellant having a boiling point of below 18° C. at atmospheric pressure. Generally, the propellant constitutes 50 to 99.9% w/w of the formulation whilst the active ingredient constitutes 0.1 to 20% w/w. for example, about 2% w/w, of the formulation.

The pharmaceutically acceptable carrier in such self-propelling formulations may include other constituents in addition to the propellant, in particular a surfactant or a solid diluent or both. Surfactants are desirable since they prevent agglomeration of the particles of active ingredient and maintain the active ingredient in suspension. Especially valuable are liquid non-ionic surfactants and solid anionic surfactants or mixtures thereof. Suitable liquid non-ionic surfactants are those having a hydrophile-lipophile balance (HLB, see Journal of the Society of Cosmetic Chemists Vol. 1 pp. 311-326 (1949)) of below 10, in particular esters and partial esters of fatty acids with aliphatic polyhydric alcohols. The liquid non-ionic surfactant may constitute from 0.01 up to 20% w/w of the formulation, though preferably it constitutes below 1% w/w of the formulation. Suitable solid anionic surfactants include alkali metal, ammonium and amine salts of dialkyl sulphosuccinate and alkyl benzene sulphonic acid. The solid anionic surfactants may constitute from 0.01 up to 20% w/w of the formulation, though preferably below 1% w/w of the composition. Solid diluents may be advantageously incorporated in such self-propelling formulations where the density of the active ingredient differs substantially from the density of the propellant; also, they help to maintain the active ingredient in suspension. The solid diluent is in the form of a fine powder, preferably having a particle size of the same order as that of the particles of the active ingredient. Suitable solid diluents include sodium chloride, sodium sulphate and sugars.

Formulations of the present invention may also be in the form of a self-propelling formulation wherein the active ingredient is present in solution. Such self-propelling formulations may comprise the active ingredient, propellant and co-solvent, and advantageously an antioxidant stabiliser. Suitable co-solvents are lower alkyl alcohols and mixtures thereof. The co-solvent may constitute 5 to 40% w/w of the formulation, though preferably less than 20% w/w of the formulation. Antioxidant stabilisers inay be incorporated in such solution-formulations to inhibit deterioration of the active ingredient and are conveniently alkali metal ascorbates or bisulphites. They are preferably present in an amount of up to 0.25% w/w of the formulation.

Formulations of the present invention may also be in the form of an aqueous or dilute alcoholic solution, optionally a sterile solution, of the active ingredient for use in a nebuliser or atomiser, wherein an accelerated air stream is used to produce a fine mist consisting of small droplets of the solution. Such formulations usually contain a flavouring agent such as saccharin sodium and a volatile oil. A buffering agent such as sodium metabisulphite and a surface-active agent may also be included in such a formulation which should also contain a preservative such as methylhydroxybenzoate.

Other formulations suitable for nasal administration include a powder, having a particle size of 20 to 500 microns, which is administered in the manner in which snuff is taken, ie by rapid inhalation through the nasal passage from a container of the powder held close up to the nose.

In addition to the aforementioned ingredients, the formulations of this invention may include one or more additional ingredients such as diluents, buffers, flavouring agents, binders, surface active agents, thickeners, lubricants, preservatives eg methylhydroxybenzoate (including anti-oxidants), emulsifying agents and the like. A particularly preferred carrier or diluent for use in the formulations of this invention is a lower alkyl ester of a C₁₈ to C₂₄ mono-unsaturated fatty acid, such as oleic acid, for example ethyl oleate. Other suitable carriers or diluents include capric or caprylic esters or triglycerides, or mixtures thereof, such as those caprylic/capric triglycerides sold under the trade name Miglyol, eg Miglyol 810.

Because these compounds antagonise the function of CCK in animals, they may also be used as feed additives to increase the food intake of animals, such as in a daily dosage of from about 0.05 to 50 mg/kg of body weight.

The invention will now be further described by way of example only.

The compounds of formula (I) can be prepared by reaction of appropriately-substituted furan-2(5H)-ones with the corresponding amine, as illustrated in scheme 1 below.

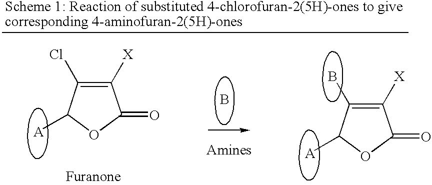

In scheme 1, A corresponds to the substituent R in formula (I) above and B corresponds to NR¹R² in formula (I) above. The compounds of the invention may be prepared by assembling a chemical library of the components of the general formula (I), such as, as follows:

Starting material are the commercially available mucochloric acid, mucobromic acid and furfural. Furfural can be converted into 5-hydroxy-4-chloro-2(5H)-furanone according to published methods.

The first step is the preparation of a sublibrary of 3,4-dihalogenated 2(5H)-furanones.

Preparation of Building Blocks/sublibrary

Formation of Furan-2(5)-One Building Blocks

Mowry (Mowry, D. T., Muchloric acid I: Reactions of the pseudo-acid group. J. Am. Chem. Soc. 1950, 2535-3537; Muchloric acid II. Reactions of the aldehyde group. J. Am. Chem. Soc. 1953, 1909-1910) stated that mucochloric acid is thought to be in the half aldehyde state of dichloromaleic acid and is thought to exist in the open and closed ring forms (Scheme 2)

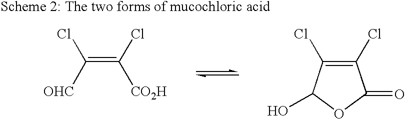

Two naming systems are mainly used, based on the two core names, furanone and butenolide, with the term furanone being preferred. In recent years there has been much interest focused on furan-2(5H)-ones because of their wide occurrence in a variety of biologically active products and their use as valuable synthetic intermediates (Lattmann, E., Hoffmann, H. M. R. From tetronic acid and furfural to C(4)-halogenated, vinylated and formylated furan-2(5H)-ones and their 5-alkoxy derivatives. Synthesis, 1996, 155-163; Hoffmann, H. M. R., Gerlach, K., Lattmann, E. New bicyclic conjugates of three- and five-membered heterocycles with 5-alkoxyfuran-2(5H)-ones Synthesis, 1996, 164-170. Lattmann, E., Coombs, J., Hoffmann, H. M. R. Pyranofuranones via lewis acid mediated hetero-Diels-Alder reactions of 4-furan-2(5H)-ones. Synthesis, 1996, 171-177.)

Mucochloric acid, is derived from furfural, which is obtained from biomass. It is a relatively inexpensive, readily available compound. The bromo-compounds, although known, do not appear to be used by organic chemists, probably due to the toxicity of the dibromno functionality.

Once the 5-subsituted-furanones have been formed, nucleophiles containing nitrogen were reacted at the 4-position. It was noted these reactions proceed in good yields and the products were relatively stable and easy to isolate.

The reactions that mucochloric acid undergoes can be divided into two sets, depending on the two tautomeric forms the molecule is thought to exist. Mowry divided the different reactions that occur in the molecule, into two groups;
(i) Reactions of the aldehyde group and
(ii) Reactions of the pseudo-acid group
   Reactions of the Aldehyde Group
   1 Condensation (Aldol) Reactions

Mucochloric acid can be reacted with compounds containing a reactive methylene and hydrogens a to a carbonyl, nitro or a cyano group. This is achieved in a cold alkaline solution to form 3,4-dichloro-(2)-furanones substituted in the 5-position. Attempts to react the aldehyde moiety of mucochloric acid under less basic or acidic conditions were successful also. This working approach, with acetophenone, was expanded to react substituted acetophenones to produce novel 3,4-dichloro-(2)-furanones, as illustrated in scheme 3 below.

Scheme 3: Synthesis of 3,4-dichloro-5-[2-sub-
phenyl)-2-oxoethyl]furan-2(5H)-ones

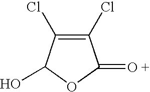

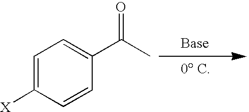

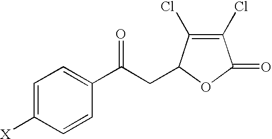

| Compound | Group | Solvent | Yield [%] |
|---|---|---|---|
| 1 | X = H | Methanol | 46.5 |
| 2 | X = OCH₃ | Methanol | 17.5 |
| 3 | X = CH₃ | Propan-2-ol | 42.8 |
| 4 | X '2 Cl | Propan-2-ol | 70.9 |

Mucochloric acid with the appropriate substituted acetophenone was dissolved in methanol/propan-2-ol, cooled to 0° C. and the base added, dropwise. The solution was allowed to stand for at least 3 hrs and then poured onto ice-water containing an excess of HCl conc. An oily precipitate formed for compound 1, while a solid formed for the remainder. This was recrystailised with ethanol. It was observed that a deviation away from methanol, a very polar solvent system towards propan-2-ol, less polar enabled products to form in good yields.

2 Preparation of 5-(3,4-dichloro-5-oxo-2,5-dihydrofuran-2-yl) imidazolidine-2,4-dione

It was decided to attempt the same condensation method with different reactants containing a cyclic alkyl functionality. An equimolar amount of mucochloric acid and hydantoin was dissolved in DCE and cooled to 0° C. A solution of NaOH was added slowly and then the mixture was allowed to stand for 4 hrs. The whole mixture was then poured into ice-water, containing an excess of HCl conc. After 45 mins the precipitate was filtered and recrystallised from dilute ethanol to give a white powder. The yield was quite low at 20%.

The reaction conditions were modified to overcome initial solubility problems. The reaction was carried out in a two-phase system of water and DCE. The novel product was fully characterized with sharp and concise ¹H and ¹³C peaks.

3 Preparation of 3,4-dichloro-5-phenylfuran-2(5H)-one

The Friedel crafts reaction conditions was utilised to prepare 3,4-dichloro-5-phenylfuran-2(5H)-one according to the published method by Semonsky et al (Semonsky, M.; Rockova, E.; Cerny, A.; Kakac, B. and Macek, K. Substanzen mit antineoplastischer Wirksamkeit IV: Einige γ-aryl-α,β-substituierte-crotolactone. Collec. Czech. Chem. Commun. 27, 1961, 1939-1954) Mucochloric acid was dissolved in benzene, which acts a both solvent and reagent, with aluminium chloride. The mixture was allowed to stir at RT for 3 days, under inert conditions. After work up a brown oil was recrystallised from ethanol for give white crystals, 54% yield. Analysis of the product was initially achieved by APCI+ mass-spectrometry, where the MS+H was just detectable and subsequently confirmed by both ¹H and ¹³C NMR spectroscopy.

4-dichlorofuran-2(5H)-one

Muccochloric acid and aluminium isoproxide was dissolved in isopropanol and refluxed using a vigreux column. Excess isopropanol was distilled off and the remaining mixture was poured into a mixture of ice-water, containing an excess of HCl conc. After the extraction and washings, the crude product was recrystallised in dilute ethanol to give a white crystalline solid in 33% yield. The product was fully characterised.

5 Preparation of 3,4-dichloro-5-oxo-2,5-dihydrofuran-2-yl amides

In a new approach various amides were reacted with mucochloric acid, under refluxing conditions with a trace of acid. Amides are generally much less reactive than acid chlorides, anhydrides and esters. The amide linkage is stable enough to serve as a basic unit. It was found that amide formation proceeded via the aldehyde group of mucochloric acid, rather than the pseudo acid.

Scheme 4: Symthesis of amide derivatives from muchochloric acid

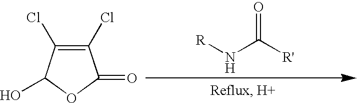

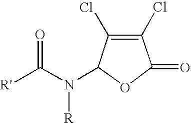

| Compound | R | R′ | Yield [%] |
|---|---|---|---|
| 5 | —CH₃ | —H | 10.5 |
| 6 | —(CCH₃)₃ | —H | 11.9 |
| 7 | —CH₃ | —CH₃ | 6.0 |
| 8 | —CH₂C₆H₅ | —H | 15.0 |
| 9 | —C₆H₅ | —H | 48.5 |

Amides have a lower bascity than amines, because they are resonance stabilised, due to the lone pair of electrons. However, an amide protonated on its nitrogen lacks this resonance stabilisation and can react via an nucleophilic subsitution type reaction (Scheme 4). Geometeric isomers were obtained (cis & trans) with these compounds. These conformational isomers may be easily inter-converted by rotation about the bond. The staggered, low energy conformation is more favourable as shown by the NMR data (¹H and ¹³C), ratio 3:1 (trans:cis). Although the staggered, trans isomer is more favourable, the eclipsed, cis-high energy conformation is formed due to the high temperature reaction conditions.

The various reactions of the aldehyde group are summarised in scheme 5 below. 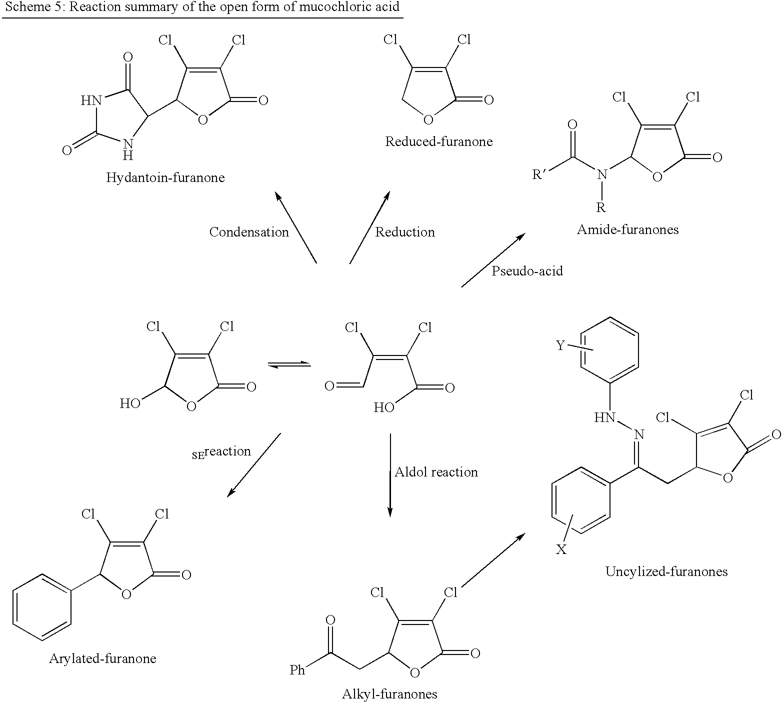
Reactions of the Pseudo-Acid Group (Closed Ring Furanone)

Refluxing mucochloric acid with various reagents resulted in the formation of pseudo esters, anhydrides, acid chlorides, which were all in the cyclic form (Scheme 6).

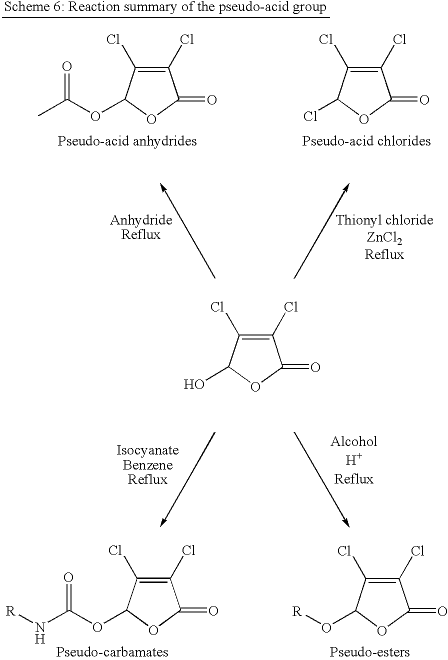

A large number of alcohols, which included: methanol, ethanol, isopropanol, n-butanol, 1-nanonol, menthol, cetyl, vinyl acetate, allyl and propargyl were included as building blocks. Phenyl and naphthyl were the two carbamates chosen.

By the above methods, the following compounds were prepared: 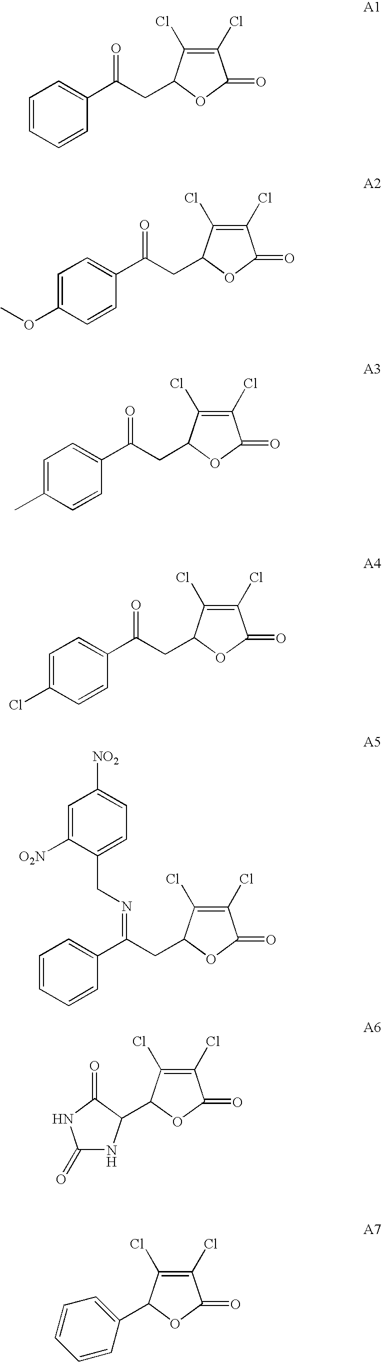 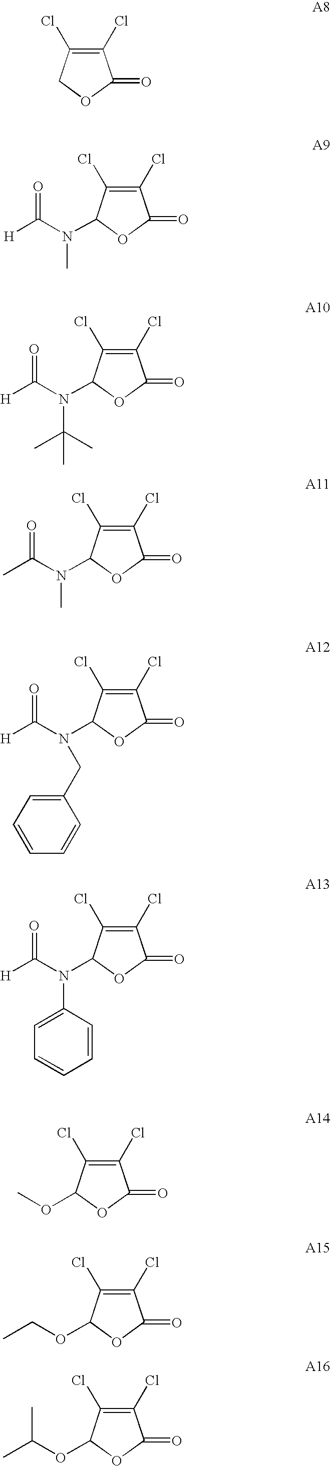 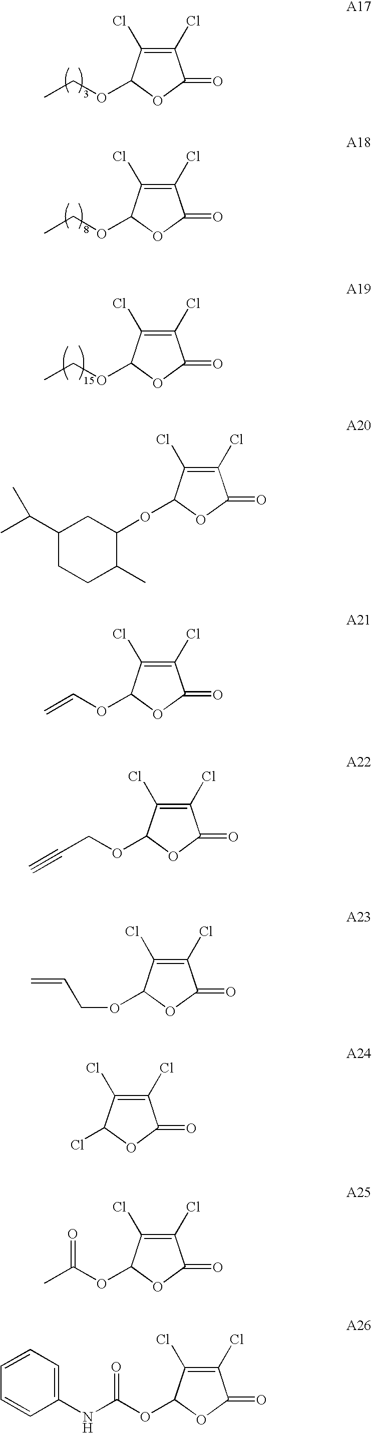 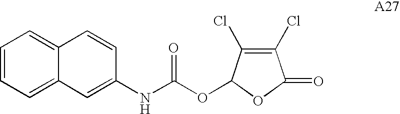
Where Aₘ denotes substituent R in fonnula (I).
Preparation of a chemical library of 4-substituted-amino-furan-2-(H)-ones

The furanone building blocks (containing R=A₁ to A₂₇) were each reacted with the amines specified in Table 1:

TABLE 1
Amines (B)

B₁ = 4-aminoantipyrine
B₂ = Indoline
B₃ = Benzimidazole
B₄ = m-Toluidine
B₅ = sec-Butylamine
B₆ = Benzylmethylamine
B₇ = Dimethylmorpholine
B₈ = N-Phenylpiperazine
B₉ = Pyrrolidine
B₁₀ = n-Dodecylamine
B₁₁ = 4-Phenylpiperidine
B₁₂ = n-Butylamine
B₁₃ = Benzylamine
B₁₄ = Aniline
B₁₅ = Benzylpiperazine
B₁₆ = 4-Amino-1-benzylpiperidine
B₁₇ = Aminopropylmorpholine
B₁₈ = 2-Chlorobenzylamine
B₁₉ = Dibenzylamine
B₂₀ = 2,6-Dimethylpiperidine
B₂₁ = N,N′-Isopropylcyclohexylamine
B₂₂ = Phenethylamine
B₂₃ = 3,5-Dimethyl pyrazole
B₂₄ = Ethyl-1-piperazine carboxylate
B₂₅ = 4-(3-Phenylpropyl) piperidine
B₂₆ = 3-Methyl pyrazole

The reaction preferably takes place in a universal solvent to dissolve all reactants; it is preferably sufficiently nucleophilic to aid product formation. A suitable such solvent is dimethyl formamide (DMF). A 12-test-tube reaction carousel or aluminium blocks may be used for the chemical reaction. The appropriate furanone building block from FIG. 1is preferably reacted with three equivalents of amine for the construction of a chemical library. The reaction mixtures are suitably heated and stirred at elevated temperatures up to 60 ° C. and may be left up to overnight. Leaving the mixtures longer causes the product to decompose into sticky black liquid. TLC analysis may be used to monitor the reaction. In general, product formation is optimal after 15-20 hours. Excess water is then added to each test tube/ vial and allowed to stand, such as for 30 minutes. The work-up phase removes any excess amine in the mixture.

Three different isolating techniques can be employed:

(1) If the target compound precipitates out of solution, it can be washed with water and dried to give a pure compound. This method is suitable to isolate isopropy-substituted furanone analogues.

(2) The target compound can be extracted with dichloromethane, the organic phase washed with water, then with diluted HCl (pH 5). The organic layer is dried and removed in vacuo. This method can be used to isolate lipophilic group-substituted furanone analogues.

(3) In addition to-method (2), chromatographic separation can be used (Preparative TLC), with either 100% ether or 10% MeOH in ether as the mobile phase. This is a suitable technique for isolating the majority of the compounds.

Examples of compounds of formula (I) preparable by this technique include the following:—

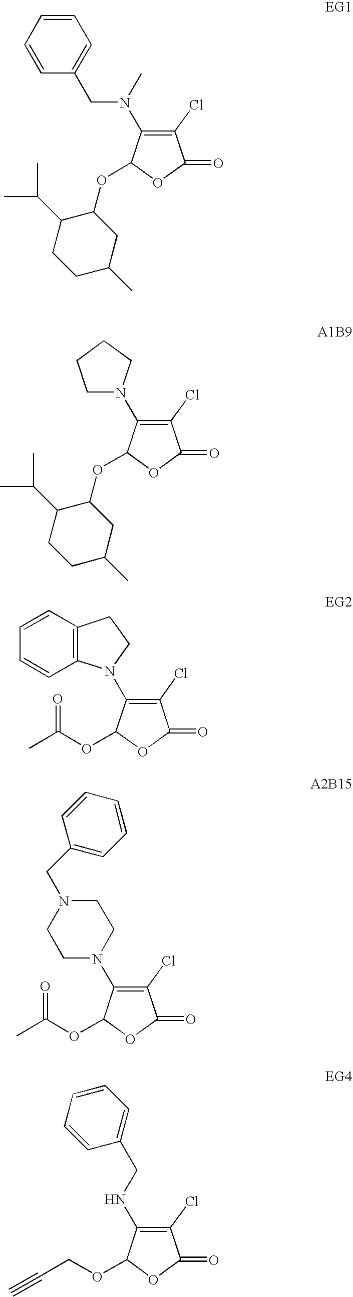

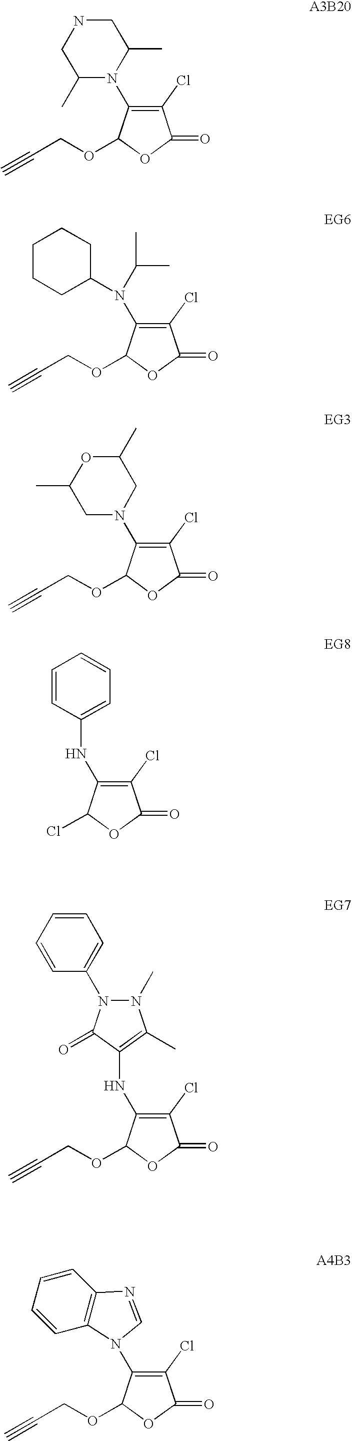

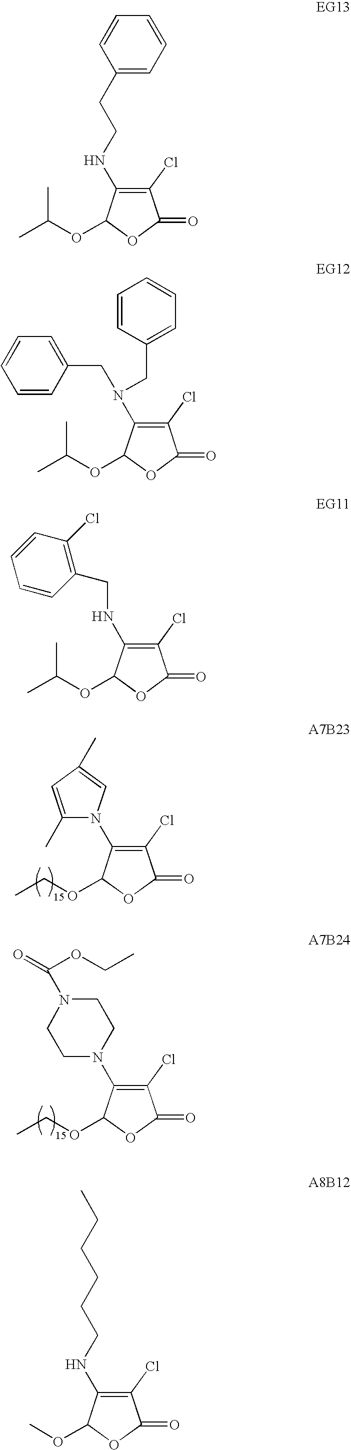

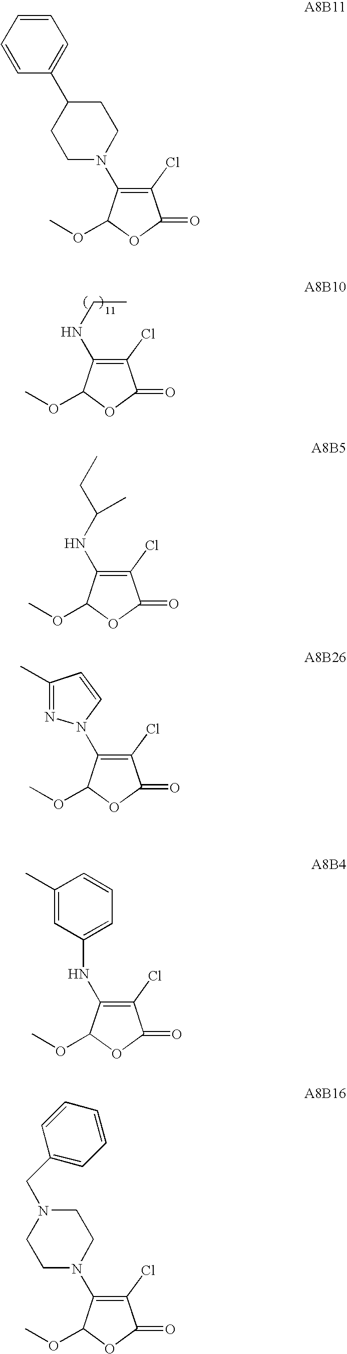

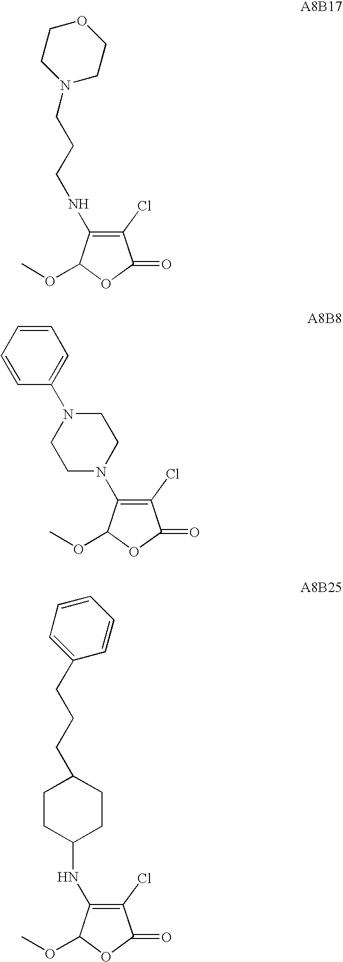

In general, a selected number of 4-substituted amino-furan-2-(H)-ones exhibited a range of modest to high CCK antagonist activity. Large, bulky substituents on the 4-position are not preferred, but smaller ligands exert excellent receptor affinity.

A second combinatorial library was constructed using the selected building blocks and amines such as methylarine, ethylamine, n-propylamine, n-butylamine, sec. Butylamine, n-amylamnine, hexylamine, decylamine, dodecylamine, 1-ethynylcyclohexylamine, N-cyclohexylethylamine, N-cyclohexyl-isopropylamine, benzimidazole, diisopropylamine, cyclohexylamine, t-butylamine, benzylamine, phenylethylamine, 3-dimethylamninopropylaamine, 4-(2-aminoethyl)-morpholine, dibutylaminopropylamine,1-(2-aminoethyl)pyrolidine, 2-dibutylaminoethylamine, diethylethylendiamine, 1-(2-aminoethyl)-piperidine, m-anisidine, 3-chloroaniline, 3-di-n-butylaamino-propylamine, 1-3-aminopropyl-2-methylpiperidine, 4-(3-aminopropyl)-morpholine, N,N-diethyl-1,3-propandiamine, N,N-dimethyl-ethylendiamine, 2,3-dimethylaniline, 3,4-dimethylaniline, 2-chlorobenzylamiine, N-ethyltoluidine, 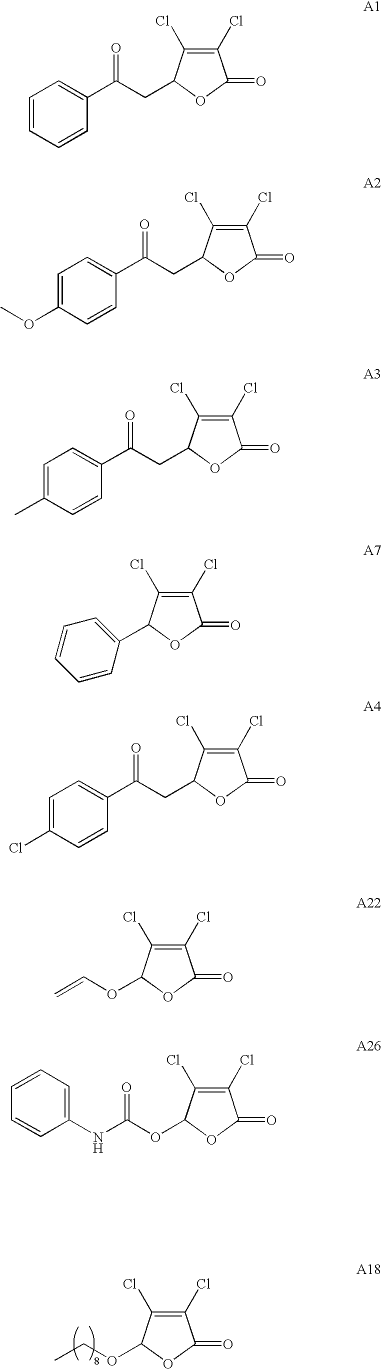 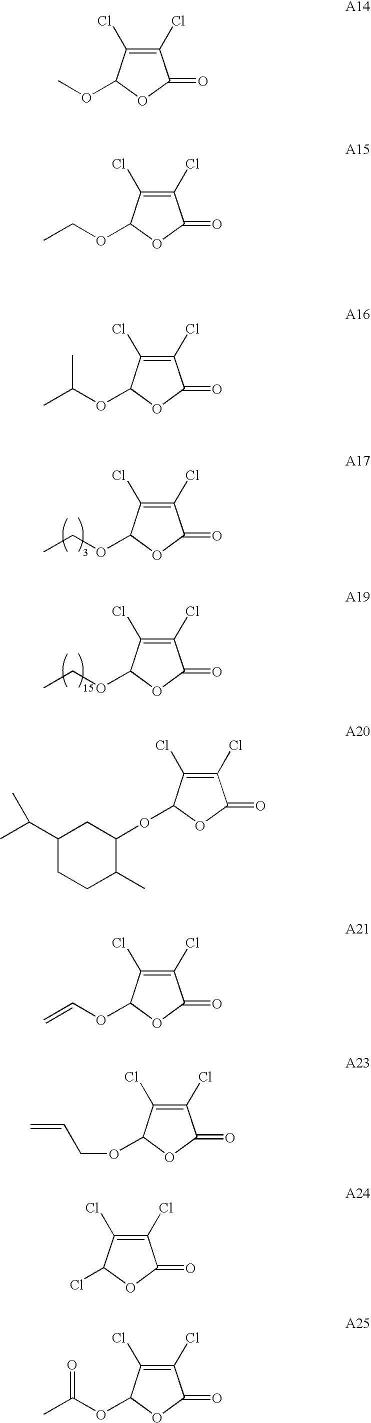
Overviewv of furanones for library II

Structures of the most active ligands from the 4substituted amino-furan-2-(H)-one series, being Examples 4, 6 and 15, 16 and 17.

| | |
|---|---|
| 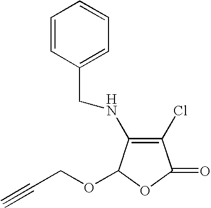 | 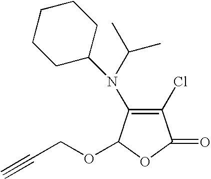 |
| Example 4 | Example 6 |

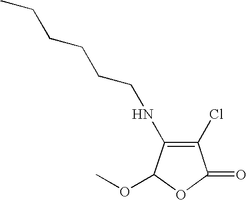

Example 15

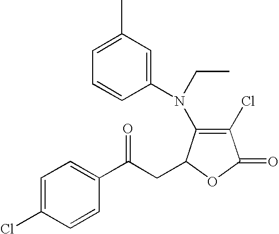

Example 16

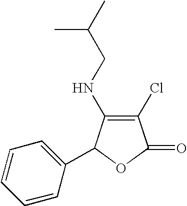

Example 17

| | Example 4 | Example 6 | Example 15 |
|---|---|---|---|
| CCK_{B} (nM) | 14 | 8 | 12 |
| CCK_{A} (nM) | 9 | 6 | 8 |
| Selectivity | 1.5 | 1.3 | 1.5 |
| | Example 16 | Example 17 | |
| CCK_{B} (nM) | 19 | 11 | |
| CCK_{A} (nM) | 8 | 7 | |
| Selectivity | 2.3 | 1.6 | |

The propargyl group is supposed to mimics an arylated system. The acetal functionality in the 5-position is in principle unstable and its removal results in a large enhancement of the chemical stability. 4-Aminofuranones of 5-arylated furanones, based on building block A7 [example 17], and 4-aminofuranones of the ketone series A1-A4 furnished ligands with a high chemical stability and a high binding affinity.

### EXAMPLES

General Synthesis Methods

The majority of chemicals used were obtained from the laboratory and chemical stores. The remainder were ordered from Aldrich Catalogue Handbook of Fine Chemicals and Lancaster 1999/2000/2001.

Construction of the sublibrary of 5-substituted 3-4-dichloro-2(5H)-furanones

Synthesis of 5-alkoxy-2(5H)-furanones; Pseudoesters

Eric Lattmann*, D. C. Billington and Christopher A. Langley, Synthesis of Combinatorial Libraries of 3,4,5-substitutes 2(5H)-furanones. Part one: Construction of a Sub-Library of Halogenated 5-Alkoxy-2(5H)-furanones, Drug Design and Discovery, 1999, 16, 237-242 and

Eric Lattmann*, D. C. Billington and Christopher A. Langley, Synthesis of Combinatorial Libraries of 3,4,5-substitutes 2(5H)-furanones. Part two: Construction of a Library of 4-Amino-5-Alkoxy-2(5H)-furanones. Drug Design and Discovery, 1999, 16, 243-250

Pseudocarbamates

Eric Lattmann, Derek Kinchington, Harjit Singh, Isidro Merino, Adiba Begum, Washington Ayuko and Mike J. Tisdale, Synthesis of Simple and Bis-cyclic 3,4-Dihalogenated 2(5H)-furanones and their Evaluation as Novel Cytotoxic Agents Pharm. Pharm. Lett. 2001, 11, 5-8

Ketone series, acetophenones, synthesis of 2-oxophenyl ethyl-2(5H)-furanones

Mucochloric acid (21.0 g, 0.125 mol) and (a) acteophenone and (b) 4-methoxyacetohenone were each dissolved in methanol (200 mnl) and cooled to 0° C. A solution of NaOH (8.0 g in 70 ml water, 2.5 M) was added slowly, whilst stirring at 0-5° C. After the addition of NaOH, the mixture was allowed to stand at RT for 3 hrs. The brown mixture was poured into ice-water, containing an excess of conc HCl and allowed to stand for 45 mins. A yellow oily liquid/solid was decanted and washed with water to give a crude yellow product. Refluxing from dilute ethanol gave a pure white powder.

### 3,4-dichloro-5-(-2-oxo-2-phenylethyl]furan-2(5H)-one

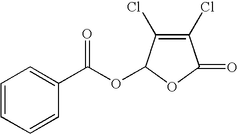 [0106] Yield: 46.5%
R_{f} (etber)=0.26
Mol. Weight: 271.1
Mol. Formula: C₁₂H₈Cl₂O₃
MS (APCI(+)): 271 (M+) m/z
IR (KBr-disc) υ max: 3010, 1773, 1683, 1648, 1210, 1033, 767 & 692 cm⁻¹.
¹H NMR (CDCl₃) 300K δ: 3.35-3.61 (m, CH₂), 5.69-5.73 (dd, CH, J=3.6 Hz), 7.45-7.52 (t, Ar-2H, J=7.8, 7.2 Hz), 7.56-7.65 (tt, Ar-H, J=7.4, 7.3 Hz), 7.91-7.95 (d, Ar-2H, J=7.1 Hz) p.p.m.
¹³C NMR (CDCl₃) 300K δ: 40.0 (CH₂), 78.0 (CH), 121.4 (C-Cl), 128.1 (2xC), 134.1, 135.7 (2xC) (Ar-C), 151.9 (C-Cl), 164.8 169.9, 193.7 (C=O) p.p.m.

### 3,4-dichloro-5-[2-(4-methoxyphenyl)-2-oxoethyl]furan-2(5H)-one

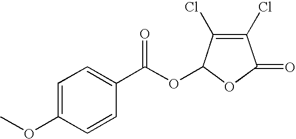 [0115] Field: 17.5%
R_{f} (ether)=0.43
Mol. Weight: 301.1
Mol. Formula: C₁₃H₁₀Cl₂O₄
MS (APCI(+)): 301 (M+) m/z
IR (KBr-disc) υ max: 3448, 1775, 1665, 1629, 1598, 1258, 1209, 1174, 1031, 983 & 744 cm⁻¹.
¹H NMR (CDCl₃) 300K δ: 3.29-3.57 (m, CH₂), 3.86 (s, CH₃), 5.68-5.72 (dd, CH, J=3.7 Hz), 6.89-6.96 (d, Ar-2H, J=9.0 Hz), 7.87-7.93 (d, Ar-2H, J=9.0 Hz) p.p.m.
¹³C NMR (CDCl₃) 300K δ: 39.6 (CH₂), 55.3 (CH₃), 78.2 (CH), 113.9 (2xAr-C), 121.2 (C-Cl), 128.7, 130.5 (2xC), 152.1 (C-Cl), 164.2 (Ar-C), 169.9, 192.0 (C=O) p.p.m.

Mucochloric acid (21.0 g, 0.125 mol) and (a) 4-methylacteophenone and (b) 4-chloroacetohenone were each dissolved in propan-2-ol (250 ml) and cooled to 0° C. A solution of NaOH (8.0 g in 70 ml water, 2.5 M) was added slowly, whilst stirring at 0-5° C. After the addition of NaOH, the mixture was allowed to stand at RT for 3 hrs. The crude precipitate was poured into ice-water containing an excess of conc HCl and allowed to stand for 45 mins. The solid was filtered, washed with water and recrystallised from dilute propan-2-ol to give a white powder.
3,4-dichloro-5-[2-(4-methylphenyl)-2-oxoethyl]furan-2(5H)-one 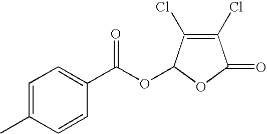
Yield: 42.8%
R_{f} (ether)=0.77
Mol. Weight: 285.1
Mol. Formula: C₁₃H₁₀Cl₂O₃
MS (APCI(+)): 285 (M+), 187 (M+) m/z
IR (KBr-disc) ,) υ max: 3420, 1783, 1677, 1631, 1602, 1368, 1183, 1019, 948 & 915 cm⁻¹.
¹H NMR (CDCl₃) 300K δ: 2.41 (s, CH₃), 3.32-3.57 (m, CH₂), 5.68-5.70 (dd, CH, J=3.6 Hz), 7.26-7.29 (d, Ar-2H, J=8.3 Hz), 7.80-7.84 (d, Ar-2H, J=8.2 Hz) p.p.m.
¹³C NlIR (CDCl₃) 300K δ: 21.6 (CH₃), 39.8 (CH₂), 78.1 (CH), 121.3 (C-Cl), 128.2 (2xC), 129.5 (2xC), 133.2, 145.1 (Ar-C), 152.0 (C-Cl), 164.8, 193.2 (C=O) p.p.m.

### 3,4-dichloro-5-[2-(4-chlorophenyl)-2-oxoethyl]furan-2(5H)-one

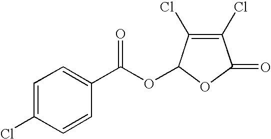 [0133] Yield: 70.9%
R_{f} (ether)=0.70
Mol. Weight: 305.5
Mol. Formula: C₁₂H₇Cl₃O₃
MS (APCI(+)): 225 (M+), 207 (M+) m/z
IR (KBr-disc) υ max: 3430, 1777, 1687, 1633, 1584, 1390, 1203, 1087, 1027, 834 & 747 cm⁻¹.
¹H NMR (CDCl₃) 300K δ: 3.32-3.57 (m, CH₂), 5.68-5.73 (dd, CH, J=3.6 Hz), 7.45-7.50 (d, Ar-2H, J=8.7 Hz), 7.85-7.90 (d, Ar-2H, J=8.8 Hz) p.p.m.
¹³C NMR (CDCl₃) 300K δ: 40.0 (CH₂), 77.8 (CH), 121.5 (C-Cl), 129.2 (2xC), 129.5 (2xC), 133.9, 140.7 (Ar-C), 151.7 (C-Cl), 164.7, 192.5 (C=O) p.p.m.
   Synthesis of Hydrazones of the Ketones

The appropriate 3,4-dichloro-5-[2-(4-substituted-phenyl)-2-oxoethyl]furan-2(5H)-one (0.1 g, 1.0 Eq) was dissolved in ethanol (20 ml), with the appropriate hydrazine (2.5 Eq). Concentrated HCl acid (0.5 ml) was added and the mixture was refluxed for up to 20 hrs. The solution was allowed to cool to RT, with the precipitate being filtered, washed and dried.
3,4-dichloro-5-{2-[(Z)-2(2,4-dinitrophenyl) hydrazono]-2-phenylethylfuran-2(5H)-one 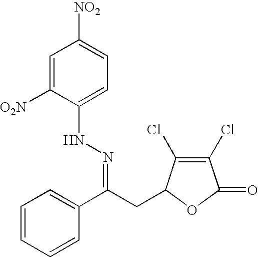
Yield: 76.1%
Mol. Weight: 451.2
Mol. Formula: C₁₈H₁₂N₄Cl₂O₆
MS (APCI(+)): 451 (M+) m/z
IR (KBr-disc). υ max: 3461, 3295, 3102, 1779, 1590, 1490, 1417, 1328, 1029 & 712 cm⁻¹.
¹³H NMR (DMSO) 300K δ: 3.51-3.79 (m, CH₂), 5.72-5.77 (dd, CH, J=3.7 Hz), 7.49-7.64 (m, Ar-5H), 8.05-8.08 (d, Ar-H, J=9.5 Hz), 8.42-8.46 (dd, Ar-H, J=9.6 Hz), 8.89-8.92 (m, Ar-H), 11.27 (s, NH) p.p.m.
¹³C NMR (DMSO) 300K δ: 33.6 (CH₂), 76.2 (CH), 90.3 (C=N), 113.6, 120.2, 123.0 (2xC), 124.1, 128.7, 129.6 (2xC), 133.7, 145.7, 145.9, 150.3, 155.9 (Ar-C), 165.8 (C=O) p.p.m.
   Synthesis of Condensation Products

### 5-(3,4-dichloro-5-oxo-2,5-dihydrofuran-2-yl)imidazolidine-2,4-dione

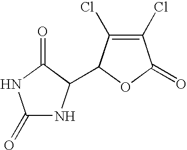

Mucochloric acid (8.45 g, 0.05 mol) and hydantoin (5.0 g, 0.65 mol) were dissolved in dichloroethane (60 ml) and cooled to 0° C. A solution of sodium hydroxide (6.0 g in 75 ml water, 2 M) was slowly added, whilst stirring at 0-5° C. After the addition of sodium hydroxide, the mixture was allowed to stand at room temperature for 4 hrs. The solution was poured into ice water containing an excess of concentrated hydrochloric acid and allowed to stand for 45 mins. The precipitate was filtered, washed with water and dried to give a crude light brown product. Recrystallisation from dilute ethanol gave a pure white powder.
Yield: 20%.
Mol. Weight: 251.0.
( Mol. Formula: C₇H₄Cl₂N₂O₄
MS (APCI(+): 251, 253 (M+1) m/z.
IR (KBr-disc) υ: 3286, 3171, 3054, 1787, 1723, 1652, 1419, 1234, 1023 & 811 cm⁻¹
¹H NMR (DMSO-d₆) 300K δ: 4.70 (t, 1H, —CH-furan, J=1.6, 1.6 Hz), 5.66 (d, 1H, —CH-hydantoin, J=1.7 Hz), 8.25 (s, —NH), 11.03 (s, —NH) p.p.m.
¹³C NMR (DMSO-d₆) 300K δ: 57.4 (CH-furan), 80.1 (CH-hydantoin),121.5 (CH-CCl), 158.2 (C-furan), 165.3 (C=O-furan), 165.3 & 172.4 (C=O-hydantoin) p.p.m.
   Synthesis of 5-arylated 2(5H)-furanones

### 3,4-dichloro-5-phenylfuran-2 (5H)-one

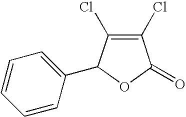

Mucochloric acid (16.38 g, 0.1 mol) was dissolved in benzene (250 ml). Powdered aluminium chloride (20 g) was slowly added to the mixture, whilst stirring. The solution was left for 3 days under inert conditions. The whole mixture was poured into an acidic-ice solution comprising of (130 g ice, 40 g HCl con) The organic phase was separated and washed with water. The benzene layer was dried over magnesium sulphate and removed in vacuo. An viscous brown oil was recrystallised from ethanol to yield white crystals.
Yield: 53.6%.
Mol. Weight: 229.0.
Mol. Formula: C₁₀H₆Cl₂O₃.
IR (KBr-disc) υ: 3526, 1772, 1625, 1287, 1228, 1025, 909, 765, 700 cm⁻¹.
MS (APCI(+)): 229, 231 (M+1) m/z.
¹H NMR (CDCl₃) 300K δ: 5.86 (s, 1H, —CH), 7.25-7.49 (m, 5H, aryl-H) p.p.m.
13C NMR (CDCl₃) 300K δ: 83.6 (CH), 121.0 (C=OCCCl), 127.1, 127.2 & 130.4 (o,m & p-aryl C), 131.6 (CH-aryl-C), 152.2 (C=OCCl), 156.3 (C=O) p.p.m.

Reduction products of Mucochloric-, mucobromic acid, 5-hydroxy-2(5H)-furanones

### 3,4-Dichlorofuran-2 (51)-one

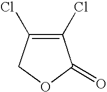

Mucochloric acid (33.8 g, 0.2 mol) and aluminium isopropoxide (50.0 g, 0.25 mol) was dissolved in isopropanol (200 ml) and refluxed using a vigreux column, until acetone ceased distilling. The excess isopropanol was removed by distillation and the mixture poured onto a mixture of ice (300 g) and concentrate hydrochloric acid (100 ml). The resulting slurry was heated to 50° C. and extracted with chloroform. After washing with water, sodium carbonate and hydrochloric acid solutions twice, the extract was distilled to give a crude product. Recrystallised from dilute ethanol to give a white solid.
Yield: 33.1%.
Mol. Weight: 152.9.
Mol. Formula: C₄H₂Cl₂O₂.
IR (KBr-disc) υ: 1781, 1631, 1442, 1351, 1243, 1013, 913, 747 cm⁻¹.
MS (APCI(+)): 153, 155 (M+1) m/z.
¹H NMR (CDCl₃) 300K δ: 4.86 (s, 1H, —CH) p.p.m.
¹³ C NMR (CDCl₃) 300K δ: 72.0 (CH), 120.6 (C=OCCCl), 149.3 (C=OCCl), 165.9 (C=O) p.pm.
   Synthesis of Pseudoamides, formamido-2(5H)-furanones

### 3-4-Dichloro-5-oxo-2,5-dihydrofuran-yl(methyl)formamide

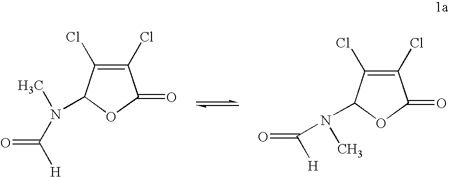 [0178] Yield: 10.5%.
R_{f} (10% MeOH/ether)=0.53.
Mol. Formula: C₆H₅Cl₂NO₃.
Mol. Weight: 210.
IR (KBr-disc) υ max: 2961, 1806, 1701, 1408, 1299, 1030, 913, 747 cm⁻¹.
MS (APCI(+)): 210 (M+1) m/z.
¹H NMR (DMSO-d₆) 300 K δ: (Isomers) 2.60, 2.84 (s, CH₃), 6.22, 6.80 (s, CH), 8.37, 8.52 (s, COH) p.p.m.
¹³C NMR (DMSO-d₆) 300 K δ: (Isomers) 24.4, 28.3 (CH₃), 81.5, 88.6 (CH), 124.0, 124.9 (C-Cl), 146.3, 147.1 (C-Cl-CO), 161.8, 162.5 (CO-O), 163.8, 167.4 (C=O) p.p.m.

### tert-butyl(3,4-dichloro-5-oxo-2,5-dihydrofuran-2-yl)formamide

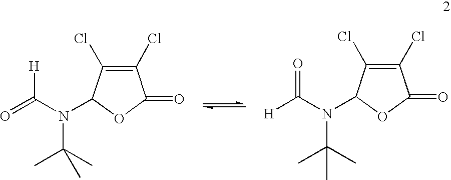 [0187] Yield: 11.9%.
R_{f} (10% MeOH/ether)=0.61.
IR (KBr-disc) υ max: 3279, 2971, 1679, 1614, 1392, 1346, 1266, 1195, 1006 cm⁻¹.
MOL. FORMULA: C₉H₁₁ClNO₃.
MOL. WEIGHT: 252.1.
MS (APCI(+)): 253 (M+1), 162, 163, 164 (M+) m/z.
¹H NMR (CDCl₃) 300 K δ: (Isomers) 1.25-1.32 (m, CH₃, 9H), 7.27, 8.14 (s, CH), 7.82, 8.89 (s, COH) p.p.m.
¹³C NMR (CDCl₃) 300 K δ: (Isomers) (28.7, 29.9, 30.17), (50.3, 51.1, 53.0) (CH₃), 61.6, 63.1 (C(CH₃)₃), 106.4 (CH), 148.8 (C-Cl), 160.8 (C-Cl-CO), 180.4 (CO-O), 192.9 (C=O) p.p.m. Cl N-(3,4-dichloro-5-oxo-2,5-dihydrofuran-2yi)-N-methylacetamide 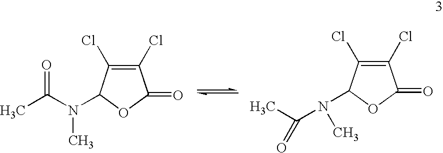
Yield: 6.0%.
R_{f} (10% MeOH/ether)=0.73.
Mol. Formula: C₇H₇Cl₂NO₃.
Mol. Weight: 224.0.
IR (KBr-disc) υ max: 3372, 2963, 1769, 1640, 1447, 1233, 1150, 1023, 946, 886, 748 cm⁻¹.
MS (APCI(+)): 224 (M+1), 182, 183, 184 (M+) m/z.
¹H NMR (DMSO-d₆) 300 K δ: (Isomers) 2.18, 2.34 (s, CH₃), 2.59, 2.79 (s, N-CH₃), 6.23 (s, CH) p.p.m.
¹³C NMR (CDCl₃) 300 K δ: (Isomers) 22.0 (CH₃), 28.9 (N-CH₃), 83.3 (CH), 124.2 (C-Cl), 148.0 (C-Cl-CO), 163.5 (CO-O), 172.3 (C=O) p.p.m.

### Benzyl(3,4-dichloro-5-oxo-2,5-dihydrofuran-2-yl)formamide

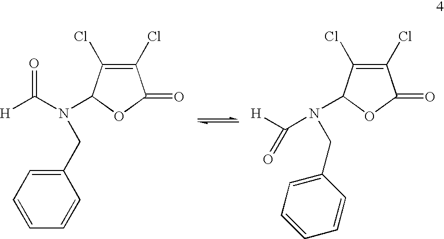 [0204] Yield: 15.0%.
R_{f} (10% MeOH/ether)=0.71.
Mol. Formula: C₁₂H₉Cl₂NO₃.
Mol. Weight: 286.1.
IR (KBr-disc) υ max: 3281, 3052, 2882, 2358, 1648, 1530, 1451, 1386, 1241, 753, 695 cm⁻¹.
MS (APCI(+)): 287 (M+1), 196, 197, 198 (M+) m/z.
¹H NMR (DMSO-d₆) 300 K δ: (Isomers) 4.32-4.34, 4.70-4.50 (sd, —CH₂—, J=6.1 Hz), 7.22-7.41 (m, phenyl-5H), 7.85, 7.90 (s, CH), 8.52, 8.90 (s, COH) p.p.m.
³C NMR (DMSO-d₆) 300 K δ: (Isomers) 41.3, 45.1 (m, CH₂), 100.4, 105.0 (s, CH), 127.4 (2xC), 127.5, 127.6, 127.8, 127.8 (2xC), 127.9, 128.8 (2xC), 128.9, 129.0 (2xC), 129.1 (Ar-C), 139.3 (C-Cl), 140.1 (C-Cl-CO), 155.7, 161.6 (CO-O), 165.5 (C=O) p.p.m.

Mucochloric acid (15.0 g, 88.8 mmol) and the relvent-amide (2: N-tert-butyl-formnamide, 3: N-methylacetamide, 4: N-benzylformamide) (133.2 mmol) were refluxed in toluene (180 ml), under a Dean stark trap, with 8-10 drops of H₂SO₄ conc. After 48-60 hrs the mnixture was cooled to room temperature. Chloroform and water was added, with the organic layer separated and washed with a further portion of water. The organic layer was dried over magnesium sulphate and removed in vacuo. A viscous crude liquid was obtained. Column chromatography (MP=10%, MeOH in ether) yielded the corresponding crystalline formamide product.

### 3,4-dichloro-5-oxo-2,5-dihydrofuran-2-yl(phenyl)formamide

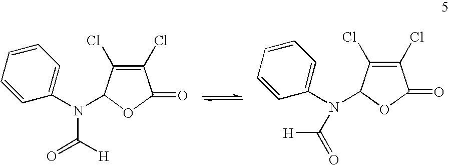

Mucochloric acid (15.0 g, 88.8 mmol) and formanilide 21.51 g, 133.2 mmol) were refluxed in toluene (180 ml), under a Dean stark trap, with 8-10 drops of H₂SO₄ conc. After 48-60 hrs the mixture was cooled to room temperature. A dark yellow precipitate was filtered, washed with toluene and dried, to give a yellow crystalline powder.
Yield: 48.5%.
R_{f} (10% MeOH/ether)=0.90.
Mol. Formula: C₁₁H₇Cl₂NO₃.
Mol. Weight: 272.1.
IR (KBr-disc) υ max: 3426, 3048, 2971, 1627, 1581, 1484, 1328, 1266, 1187, 757, 684 cm⁻¹.
MS (APCI(+)): 273(M+1) m/z.
¹H NMR (DMSO-d₆) 300 K δ: (Isomers) 7.05-7.10 (t, Ar-H, J=7.2 Hz), 7.29-7.52 (m, Ar-H), 7.66-7.69 (d, Ar-H, J=7.9 Hz), 9.22, 9.48 (s, CH), 11.81 (s, COH) p.p.m.
¹³C NMR (DMSO-d₆) 300 K δ: (Isomers) 105.8, 113.4 (CH), 122.1, 124.2 (2xC), 128.1, 128.7, 132.0, 132.8, 134.5, 134.8 (2xC) (Ar-C), 145.5(C-Cl), 152.5 (C-Cl-CO), 160.5 (CO-O), 187.4 (C=O) p.p.m.
   Construction of the Combinatorial Library

The appropriate furanone building block (compounds having R=A₁ to A₈) (0.02 g) was dissolved in DMF (20 ml) and placed into test tubes in the reaction carousel. The relevant amine (3 equivalents) was added to each tube and was allowed to stir at a temperature of 50° C. The mixtures were left overnight and monitored by TLC. Water (25 ml) was added to each mixture and allowed to stand for 30 minutes. Then:
Method 1 The precipitated compound was filtered, washed with water and dried.
Method 2 The compound was extracted with DCM and washed with dilute HCl (pH 5) and water twice. The organic layer was dried and removed in vacuo.
Method 3 The compound was extracted with DCM and washed with dilute HCl (pH 5) and water twice. The organic layer was dried and removed in vacuo. Chromatographic separation was achieved with either 100% ether or 10% MeOH in ether, as the mobile phase.

Mass spectrometric analyses was obtained by Atmospheric Pressure Chemical Ionisation (APCI), negative or positive mode, using a Hewlett-Packard 5989b quadrupole instrument. This was connected to an electrospray 59987A unit with automatic injection (Hewlett-Packard 1100 series autosampler). Samples were dissolved in HPLC grade methanol, toluene or acetonitrile.

Both Proton and Carbon NoM spectra were obtained on a Brucker AC 250 instrument, operating at 250 MHz, calibrated with the solvent reference peak or TMS.

IR spectra were plotted from KBr discs on a Mattson 300 FTIR Spectrophotometer.

Melting points were recorded from a Stuart Scientific Melting Point (SMP1) and are uncorrected.

Analytical Thin Layer Chromatography was obtained using aluminium sheets, silica gel₆₀ P254 and visualized using ultraviolet light.

Preparative chromatography was performed on 250 μm, 20×20 cm silica gel TLC plates from Aldrich.

Jencons sonomatic sonicator (SO175) was used to prepare samples for screening. All compounds for screening were prepared to 1 μM in HPLC grade DMSO.

Small scale solution syntheses were carried out on a carousel reaction station (RR 98030), comprising a 12-place carousel reaction station and reflux head, and 12×flexible tubing from Radleys, on a RCT basic hotplate from IKA Labortechnik with IKATRON ETS D3 temperature controller or using heating blocks (TECHNE Dri-block DB-3A).

### Example 1

Preparation of 4-[Benzyl(metliyl)arino]-3-chloro-5-[(2-isopropyl-5-methlylcyclohexyl)oxo] furan-2(5H)-one

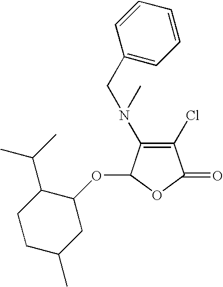

Following method 2, the title compound was prepared and identified:
R_{f} (ether)=0.33
Mol. Weight: 291.9.
Mol. Formula: C₂₂H₃₀ClNO₃.
MS (APCI(+)): 392, 394 (M+1), 254, 256 (M+) m/z.
IR (KBr-disc) υ max: 3472, 2954, 2867, 1746, 1629, 1449, 1342, 1270, 1108, 1025, 979, 738 & 699 cm⁻¹.
¹H NMR (CDCl₃) 300K δ: (isomers) 0.81-1.19 overlapping (m, CH₃), CH₂, CH, 14H), 1.60-1.66 (m, CH₂), 2.08-2.41 (mn, CH₂), 3.07 (s, CH₂), 3.10 (s, N-CH₃), 3.55-3.72 (mn, CH), 5.80 (s, CH), 7.21-7.39 (m, Ar-5H) p.p.m.
¹³C NMR (CDCl₃) 300K δ: 15.7 (2xCH₃), 20.7 (CH₃), 21.0 (CH), 23.2 (CH), 25.2 (CH₂), 31.5 (CH₂), 38.1 (N-CH₃), 42.2 (CH), 47.7 (CH), 55.4 (CH₂-Ar), 80.6 (alkyl-CH-O), 94.3 (CH-O), 97.3 (C-Cl), 127.0 (2xC), 127.8 (2xC), 135.6,135.7 (Ar-C), 156.5 (C-N), 168.3 (C=O) p.p.m.

### Example 2

Preparation of 4-ClIforo-3-(2,3-dihydro-1H-indol-1-yl)-5-oxo-2,5-dihydrofuran-2-yl acetate

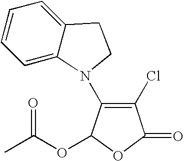

Following method 2, the title compound was prepared and identified:
R_{f} (ether)=0.83
Mol. Weight: 293.7.
Mol. Formula: C14H₁₂ClNO₄.
MS (APCI(+)): 294, 296 (M+1), 252, 254 (M+), 234, 236 (M+) m/z.
IR (KBr-disc) ™ max: 2933, 1764, 1629, 1590, 1488, 1409, 1205, 1062, 977, 911 & 752 cm⁻¹.

¹H NMR (CDCl₃) 300K δ: 1.98 (s, CH₃), 3.08-3.49 (m, CH₂), 4.39-4.49 (m, CH₂), 5.76-5.79 (d, Ar-1H, J=8.1 Hz), 6.99-7.05 (t, Ar-1H, J=7.3, 7.2 Hz), 7.12-7.18 (t, Ar-1H, J=8.0, 7.9 Hz), 7.26-7.23 (d, Ar-1H, J=8.2 Hz) p.p.m.
³C NMR (CDCl₃) 300K δ: 20.3 (CH₃), 28.5 (CH₂), 51.9 (CH₂-N), 88.4 (CH), 112.8 (Ar-C), 114.3 (C-Cl), 124.0, 125.8, 127.6, 131.9, 142.1 (Ar-C), 151.0 (C-N), 166.6, 168.9 (C=O) p.p.m.

### Example 3

Preparation of 3-Chloro-4-(2,6-dimethylmorpholin-4-yl)-5-(prop-2-ynyloxy)furan-2(5H)-one

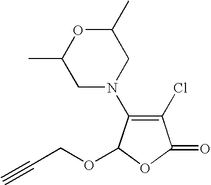

Following method 3, the title compound was prepared and identified:
R_{f} (ether)=0.53
Mol. Weight: 285.7.

Mol. Formula: C₁₃H₁₆CINO₃.
MS (APCI(+)): 286, 288 (M+1), 230, 232 (M+) mnz.
IR (KBr-disc) υ max: 3210, 2988, 2853, 1786, 1699, 1552, 1409, 1348, 1277, 1100 & 744 cm⁻¹.
¹H NMR (CDCl₃) 300K δ: 1.17 (CH₃), 1.20 (CH₃), 2.57-2.59 (t, CH=C, J=2.3, 2.4 Hz), 2.70-2.88 (m, CH), 3.44-3.48 (m, CH), 3.65-3.76 (m, CH₂), 4.09-4.34 (m, CH₂), 4.43 (s, CH₂-O), 5.95 (s, CH-O) p.p.m.

### Example 4

Preparation of 4-(Benzylamino)-3-chloro-5-(prop-2-ynyloxy)furan-2(5H)-one

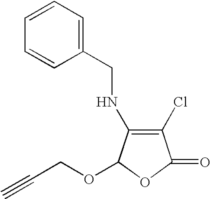

Following method 3, the title compound was prepared and identified:
R_{f} (ether)=0.51
Mol. Weight: 277.7.
Mol. Formula: C₁₄H₁₂ClNO₃.
MS (APCI(+)): 278, 280 (M+1) m/z.
IR (KBr-disc) υ max: 3380, 3283, 2358, 2338, 1752, 1646, 1455, 1326, 1123, 971 & 695 cm⁻¹.
¹H NMR (CDCl₃) 300K δ: 2.54-2.56 (t, CH=C, J=2.4 Hz), 4.44-4.47 (m, CH), 4.66 (s, CH₂), 5.20 (s, NH), 5.98 (s, CH), 7.26-7.44 (m, Ar-5H) p.p.m.

### Example 5

Preparation of 4-(4-Benzylpiperazin-1-yl) 3-chloro-5-(prop-2-ynyloxy)furan-2(5H)-one

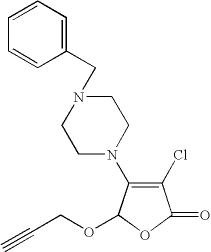

Following method 3, the title compound was prepared and identified:
R_{f} (10% MeOH/ether)=0.23
Mol. Weight: 346.8.
Mol. Fonnula: C₁₈H₁₉ClN₂O₃.
MS (APCI(+)): 347, 349(M+1) m/z.
IR (KBr-disc) υ max: 3253, 2938, 2815, 2125, 1756, 1623, 1452, 1349, 1276, 1228, 1106, 983, 742 & 698 cm⁻¹.
¹H NMR (CDCl₃) 300K δ: 2.29 (s, C=CH), 3.27-3.56 (m, CH₂-N, 8H), 3.72 (s, CH₂-Ar), 4.41 (s, CH₂-O), 5.94 (s, CH), 7.26-7.33 (m, Ar-5H) p.p.m.
¹³C NMR (CDCl₃) 300K δ: 47.4 (CH₂-O), 52.6 (CH₂-Nx2), 55.6 (CH₂-Nx2), 62.6 (CH₂-Ar), 76.8 (C=CH), 86.3 (C=CH), 94.2 (CH), 103.2 (C-Cl), 127.4, 128.3 (2xC), 129.1 (2xC), 137.0 (Ar-C), 153.9 (C-N), 168.3 (C=O) p.p.m.

### Example 6

Preparation of 3-Chloro-4-[cyclohexyl(isopropyl)amino]-5-(prop-2-ynyloxy)furan-2(5H)-one

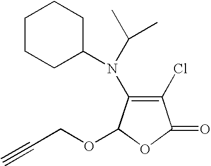

Following method 3, the title compound was prepared and identified:
R_{f} (ether)=0.58
Mol. Weight: 311.8.
Mol. Formula: C₁₆H₂₂ClNO₃.
MS (APCI(+)): 312, 314 (M+1) m/z.
IR (KBr-disc) υ max: 3440, 2927, 2362, 2338, 1702, 1636, 1552, 1447, 1128, 1098 & 1044 cm⁻¹.
¹H NMR (CDCl₃) 300K δ: overlapping 1.11-2.06 (m, CH₃, CH₂, CH, 16H), 2.09-2.16 (t, CH=C, J=2.4 Hz), 3.46-3.97 (m, CH₂, 6H), 5.99 (s, CH) p.p.m.

### Example 7

Preparation of 3-Chloro-4-[(1,5-dimethyl-2-phenyl-1,3-dihydro-3H-pyrazol-3-one)amino]-5-(vinyloxy)furan-2(5H)-one

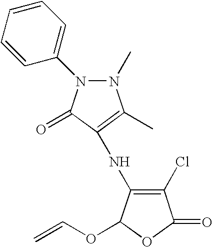

Following method 2, the title compound was prepared and identified:
R_{f} (10% MeOH/ether)=0.2
Mol. Weight: 361.8.
Mol. Formula: C₁₇H₁₆ClN₃O₄.
MS (APCI(+)): 362, 664 (M+1), 336, 338 (M+), 318, 320 (M+) m/z.
IR (KBr-disc) υ max: 3448, 2927, 2358, 1766, 1658, 1488, 1372, 1310, 1222, 1154 & 968 cm⁻¹.
¹H NMR (DMSO-d₆) 300K δ: 2.22 (s, CH₃), 2.68-2.73 (d, CH, J=13.9 Hz), 2.89-2.97 (d, CH, J=19.6 Hz), 3.36 (s, N-CH₃), 5.72 (s, CH), 6.23-6.34 (d, CH-O, J=17.8 Hz), 7.32-7.55 (m, Ar-5H), 9.25 (s, NH) p.p.m.
¹³C NMR (DMSO-d₆) 300K δ: 10.86 (CH₃), 36.0 (N-CH₃), 82.5 (CH₂), 96.3 (CH), 105.3 (C-Cl), 109.2 (C-N), 122.9, 124.3 (2xC), 129.7 (2xC) (Ar-C), 136.2 (C-CH₃), 136.4 (Ar-C), 154.7 (CH₂-O), 159.9 (C-N), 168.4, 170.2 (C=O) p.p.m.

### Example 8

Preparation of 4-Anilino-3,5-dichlorofuran-2(5H)-one

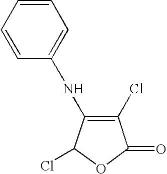

Following method 1, the title compound was prepared and identified:
R_{f} (ether)=0.62
Mol. Weight: 241.1.
Mol. Formula: C₁₀H₇Cl₂NO₂.
MS (APCI(+)): 244 (M+1) m/z.
IR (KBr-disc) υ max: 3434, 3183, 2923, 1750, 1641, 1598, 1405, 1201 & 979 cm⁻¹.
¹H NMR (CDCl₃) 300K δ: 6.25 (s, CH), 7.10-7.41 (m, Ar-5H), 9.66 (s, NH) p.p.m.

### Example 9

Preparation of 3-Chloro-4-(2,3-dihydro-1H-indol-1-yl)-5-isopropoxy-2(5H)-one

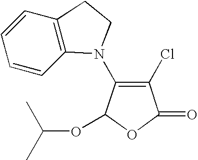

Following method 1, the title compound was prepared and identified:
R_{f} (ether)=0.58
Mol. Weight: 293.7.
Mol. Formula: C₁₅H₁₆ClNO₃.
MS (APCI(+)): 294, 296 (M+1), 252, 254 (M+) m/z.
IR (KBr-disc) υ max: 2979, 2927, 1741, 1589, 1488, 1303, 1243, 1104, 950 & 757 cm⁻¹.
¹H NMR (CDCl₃) 300K δ: 1.10 (s, CH₃), 1.24 (s, CH₃), 3.05-3.36 (m, CH₂), 4.03-4.13 (q, CH-(CH₃)₂, J=6.2 Hz), 4.27-4.49 (m, CH₂), 6.29 (s, CH-O), 6.95-7.04 (m, Ar-2H), 7.15-7.26 (m, Ar-2H) p.p.m.
¹³C NMR (CDCl₃) 300K δ: 22.0 (CH₃), 23.2 (CH₃), 28.7 CH-(CH₂)₂), 51.2 (CH₂), 73.9 (CH₂), 97.2 (CH), 113.9 (C-Cl), 123.5 (2xC), 125.4, 127.0 (2xC), 131.9 (Ar-C), 142.6 (C-N), 152.2 (C=O) p.p.m.

### Example 10

Preparation of 4-[Benzyl(methyl)amino]-3-chloro-5-isopropoxyfuran-2(5H)-one

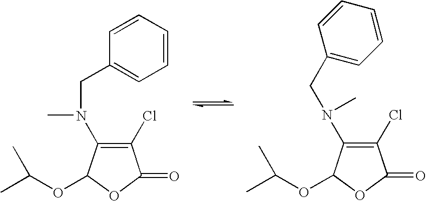

Following method 1, the title compound was prepared and identified:
R_{f} (ether)=0.50
Mol. Weight: 295.8.
Mol. Formula: C₁₅H₁₈ClNO₃.
MS (APCI(+)): 296, 298 (M+1), 254, 256 (M+) m/z.
IR (KBr-disc) υ max: 2971, 2919, 1749, 1636, 1461, 1407, 1345, 1322, 1208, 1110, 981, 958 & 752 cm⁻¹.
¹H NMR (CDCl₃) 300K δ: (isomers) 1.18-1.20 (d, CH₃, J=6.2 Hz), 1.26-1.28 (d, CH₃, J=6.2 Hz), 1.82 (s, —CH₂—), 2.95, 3.19 (s, N-CH3), 4.05 (q, CH-(CH₃) J=6.2 Hz), 4.69 (s, CH-O), 7.23-7.42 (m, Ar-H) p.p.m.
¹³C NMR (CDCl₃) 300K δ: 21.5 (CH₃), 23.2 (CH₃), 37.8 (N-CH₃), 55.4 (CH₂), 72.9 (CH-(CH₃)₂), 96.0 (CH-O), 162.3 (C-Cl), 127.2, 127.9 (2xC), 128.9 (2xC), 135.6 (Ar-C), 155.9 (C-N), 168.4 (C=O) p.p.m.

### Example 11

Preparation of 3-Chloro-4-[(2-chlorobenzyl)amino]-5-isopropoxy-4-2(SH)-oue

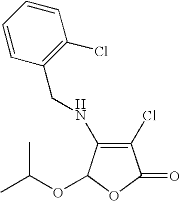

Following method 1, the title compound was prepared and identified:
R_{f} (ether)=0.51
Mol. Weight: 316.2.
Mol. Formula: C₁₅H₁₈ClN₂O₃.
MS (APCI(+)): 316, 317, 318 (M+1), 274, 275, 276 (M+), 125, 127 (M+) m/z.
IR (KBr-disc) υ max: 3280, 3085, 2975, 1739, 1644, 1556, 1430, 1305, 1230, 944 & 744 cm⁻¹.
¹H NMR (CDCl₃) 300K δ: 1.20-1.23 (d, CH₃), J=6.2 Hz), 1.24-1.27 (d, CH₃, J=6.2 Hz), 4.03-4.13 (q, CH-(CH₃)₂, J=6.2 Hz), 4.68-4.82 (m, CH₂), 4.93 (s, NH), 5.79 (s, CH-O), 7.26-7.43 (m, Ar-4H) p.p.m.
¹³C NMR (CDCl₃) 300K δ: 21.7 (CH₃), 23.1 (CH₃), 45.2 (CH₂), 73.4 CH-(CH₂)₂), 95.8 (CH-O), 102.3 (C-Cl), 127.3, 128.7, 129.4, 129.8, 132.9, 134.6 (Ar-C), 156.1 (C-N), 168.7 (C=O) p.p.m.

### Example 12

Preparation of 3-Chloro-4-(dibenzylamino)-5-isopropoxyfuran-2(5H)-one

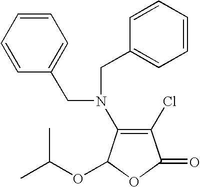

Following method 1, the title compound was prepared and identified:
R_{f} (ether)=0.64
Mol. Weight: 371.9.
Mol. Formula: C₂₁H₂₂ClNO₃.
MS (APCI(+)): 372, 374 (M+1), 330, 332 (M+) m/z.
IR (KBr-disc) υ max: 3309, 3216, 3060, 2821, 2802, 1722, 1698, 1560, 1272, 1213, 1063 & 748 cm⁻¹.
¹H NMR (CDCl₃) 300K δ: 1.08-1.10 (d, CH₃, J=6.2 Hz), 4.03-4.18 (q, CH, J=6.2 Hz), 4.64 (s, CH₂, 4H, 5.85 (s, CH-O), 7.19-7.49 (m, Ar-H) p.p.m.

### Example 13

Preparation of 3-Chloro-5-isopropoxy-4-(phenethylamine)furan-2(5H)-one

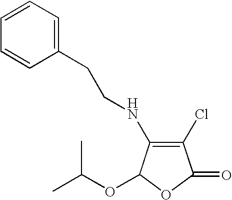

Following method 1, the title compound was prepared and identified:
R_{f} (ether)=0.56
Mol. Weight: 295.8.
Mol. Formula: C₁₅H₁₈ClN₂O₃.
MS (APCI(+)): 296, 298 (M+1), 254, 256 (M+) m/z.
IR (KBr-disc) υ max: 3274, 3073, 2971, 1733, 1644, 1556, 1337, 1284, 1241, 1008, 900 & 744 cm⁻¹.
¹H NMR (CDCl₃) 300K δ: 1.25 (s, CH₃), 1.27 (s, CH₃), 2.86-2.94 (m, CH₂), 3.70 (s, CH₂), 4.00-4.15 (m, CH), 5.03 (s, NH), 5.54 (s, CH), 7.18-7.35 (m, Ar-5H) p.p.m.
¹³C NMR (CDCl₃) 300K δ: 21.9 (CH₃), 23.2 (CH₃), 37.0 (CH₂-Ar), 44.8 (CH₂-N), 73.3 (CH-(CH₃)₂), 95.9 (CH), 100.2 (C-Cl), 126.9 (2xC), 128.4, 128.8 (2xC), 137.5 (Ar-C), 150.0 (C-N), 168.2 (C=O) p.p.m.

### Example 14

Preparation of 3-Chloro-4-[(1,5-dimethyl-2-phenyl-1,2-dihydro-3H-pyrazol-3-one)amino-5-methoxy-furan-2(5H)-one

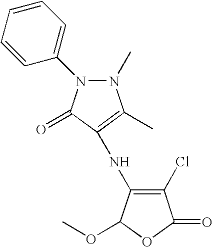

Following method 2, the title compound was prepared and identified:
R_{f} (ether)=0.78
Mol. Weight: 349.8
Mol. Fonnula: C₁₆H₁₆ClN₃O₄.
MS (APCI(+)): 350, 351 (M+1), 318, 320 (M+) m/z.
IR (KBr-disc) υ max: 3432, 3173, 3065, 2919, 1756, 1664, 1488, 1401, 1314, 1228, 1141, 1018 & 952 cm⁻¹.
¹HNMR (DMSO-d₆) 300K δ: 2.23 (s, CH₃), 3.10 (s, N-CH₃), 3.51 (s, CH-O), 5.99 (s, CH), 7.33-7.55 (m, Ar-5H), 9.09 (s, NH) p.p.m.
¹³C NMR (DMSO-d₆) 300K δ: 11. 1 (CH₃), 35.9 (N-CH₃), 56.4 (CH₂-O), 98.5 (CH), 102.0 (C-Cl), 107.7 (C-N), 124.7 (2xC), 127.3, 129.7 (2xC) (Ar-C), 135.0 (C-CH₃), 135.3 (Ar-C), 154.8 (C-N), 161.6, 168.2 (C=O) p.p.m.

### Example 15

Preparation of 3-Chloro-4-(hexylamino)-5-methoxyfuran-2(5H)-one

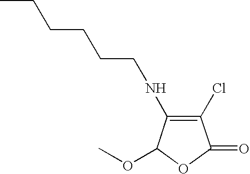

Following method 3, the title compound was prepared and identified:
R_{f} (ether)=0.46
Mol. Weight: 219.7.
Mol. Formula: C₉H₁₄ClNO₃.
MS (APCI(+)): 220, 222 (M+1) m/z.
IR (KBr-disc) υ max: 3326, 2933; 2855, 1746, 1646, 1445, 1475, 1125, 1013 & 961 cm⁻¹.
¹H NMR (CDCl₃) 300K δ: 0.83-0.86 (m, CH₃), 1.25-1.28 (m, CH₂), 1.56-1.61 (m, CH₂), 3.37-3.46 (m, CH₂), 3.57 (s, CH₃-O), 5.32 (s, CH-O), 5.65 (NH) p.p.m.

### Example 16

3-Chloro-5-[2-(4-chlorophenyl)-2-oxoethyl]-4-(ethyl-3-methylanilino) furan-2(5H)-one

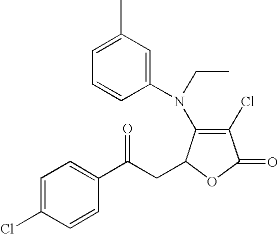

Following method 3, the title compound was prepared and identified:
Mol. Weight: 404.3.
Mol. Formula: C₂₁H₁₉Cl₂NO₃.
MS (APCI(+)): 404 (M+1), 368 (M+) m/z.
IR (KBr-disc) υ max: 2927, 2358, 1765, 1675, 1544, 1619, 1592, 1247, 1089, 1027 & 840 cm⁻¹.
¹H NMR (CDCl₃) 300K δ: 1.24-1.30 (t, CH₃, J=7.1, 7.2 Hz), 2.42 (s, Ar-CH₃, 3.66-3.75 (q, CH₂, J=7.2 Hz), 5.22-5.27 (d, CH₂-CO, J=9.9 Hz), 7.12 (s, CH), 7.26-7.43 (m, Ar-8H) p.p.m.
¹³C NMR (CDCl₃) 300K δ: 10.9 (CH₃), 21.4 (CH₃-Ar), 51.6 (CH₂-CO), 89.1 (CH₂), 104.2 (CH), 124.4 (C-Cl), 127.9, 128.6 (2xC), 129.1 (2xC), 129.3, 130.2, 136.0, 139.4, 140.7, 143.2 (Ar-C), 151.5 (C-N), 158.2 (C-N), 168.1 (C=O), 186.4 (C=O) p.p.m.

### Example 17

3-Chloro-4-(isobutylamino)-5-phenyl furan-2(5H)-one

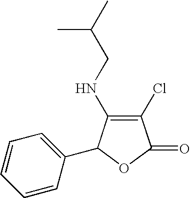

Following method 3, the title compound was prepared and identified:
Mol. Weight: 265.7.
Mol. Formula: C₁₄H₁₆ClNO₂.
MS (APCI(+)): 266, 268 (M+1), 227, 228 (M+), 193, 195 (M+) m/z.
IR (KBr-disc) υ max: 3413, 3063, 2962, 2929, 1695, 1683, 1452, 1241, 1025, 927, 757 & 699 cm⁻¹.
¹H NMR (CDCl₃) 300K δ: 0.73 (s, CH₃), 0.76 (s, CH₃), 2.65-2.75 (m, CH), 3.13-3.20 (m, CH₂), 6.08 (s, CH), 7.30-7.45 (m, Ar-5H) p.p.m.
¹³C NMR (CDCl₃) 300K δ: 20.4 (CH₃), 27.4 (CH₃), 47.6 (CH-(CH₃)₂), 93.1 (CH₂), 121.5 (C-Cl), 126.2 (2xC), 128.7 (2xC), 135.2, 138.4 (Ar-C), 155.8 (C-N), 168.5 (C=O) p.p.m.
   Biological Evaluation—^{[125]}I-CCK-8 Receptor Binding Essay:

CCK_{A} and CCK_{B} receptor binding assays were performed, by using guinea pig cerebral cortex (CCK_{B}) or rat pancreas (CCK_{A}). Male guinea pig brain tissues were prepared according to the modified method described by Saita et al, [(1994), Characterization of YM022: its CCKB/gastrin receptor binding profile and antagonism to CCK-8-induced Ca2+mobilization., Eur. J. Pharmacol.,269, 249-254]. Pancreatic membranes were prepared in a similar way but by Charpentier et al, [(1988), Cyclic cholecystokinin analogues with high selectivity for central receptors., Proc Natl Acad Sci USA, 85, 1968-1972]. The in vivo CCK binding assay: Tissues were homogenized in ice cold sucrose (0.32 M, 25 ml) for 15 strokes at 500 rpm and centrifuged at 13000 rpm for 10 mins. The supernatant was re-centrifuged at 13000 rpm for 20 mins. The resulting pellet was re-dispersed to the required volume of buffer at 500 rpm and stored in aliquots at 70° C.

Binding was achieved using a radioligand ¹²⁵I-Bolton-Hunter labeled CCK, NEN at 25 pM. The samples were incubated {with membranes (0.1 mg/ml) } in 20 mM Hepes, 1 mM EGTA, 5 mM MgCl₂, 150 mm NaCl, 0.25 mg/ml bacitracin at pH 6.5 for 2 hrs at RT and then suspended by centrifugation at 1100 rpm for 5 minutes. The membrane pellets were washed twice with water and the bound radioactivity was measured in a Packard Cobra Auto-gamma counter (B5005). All binding assays were carried out with L-363, 260 as an internal non-specific standard. Controls (no compound) were also added. All samples were made in duplicate and repeated twice. All compounds were initially screened for percentage inhibition at 20 μm. Samples showing an average inhibition of <35% were diluted to 2 μm and re-screened and if active diluted again. This enabled the calculation of IC₅₀'s of the most active compounds.

In vitro activity (IC₅₀'s) against CCK_{B} of 4-substituted amino-furan-2-(H)-ones are as shown in Table 2.

Where Aₘ and Bₙ denote the substituents R and R₁R₂N in fonnula (I) as indicated previously. As can be seen from Table 2, the majority of compounds exhibited modest to excellent activity in the nanomolar range.

TABLE 2

| | | Activity | | | Activity |
|---|---|---|---|---|---|
| | MS | CCK-B | | MS | CCK-B |
| Example | [m/z] | [μM] | Example | [m/z] | [μM] |
| 1 | 392 | 0.31 | A₇B₃ | 475 | 1.2 |
| A₁B₇ | 386 | 10.3 | A₇B₁₅ | 533 | 13.9 |
| A₁B₉ | 342 | 2.2 | A₇B₁₈ | 499 | 0.24 |
| 2 | 294 | >20 | A₇B₂₃ | 452 | 0.86 |
| A₂Bp₁₅ | 351 | >20 | A₇B₂₄ | 515 | 12.4 |
| A₃B₂ | 290 | >20 | 14 | 350 | 18 |
| 3 | 286 | 12.6 | A₈B₂ | 266 | 1.5 |
| 4 | 278 | 0.014 | A₈B₃ | 265 | 2.3 |
| 5 | 347 | >20 | A₈B₄ | 254 | 2.5 |
| A₃B₂₀ | 284 | 11.2 | A₈B₅ | 220 | 1.2 |
| 6 | 312 | 0.008 | A₈B₇ | 262 | 15 |
| 7 | 362 | 3.3 | A₈B₈ | 309 | 14 |
| A₄B₂ | 278 | 18.5 | A₈B₁₀ | 332 | 2.5 |
| A₄B₃ | 277 | >20 | A₈B₁₁ | 308 | 3 |
| A₅B₁ | 355 | >20 | 15 | 220 | 0.012 |
| A₅B₂ | 270 | >20 | A₈B₁₃ | 254 | 1 |
| 8 | 244 | 16 | A₈B₁₅ | 323 | 14 |
| 9 | 2294 | 2.5 | A₈B₁₆ | 323 | 16 |
| 10 | 296 | 12 | A₈B₁₇ | 291 | 15 |
| A₆B₁₅ | 351 | >20 | A₈B₂₂ | 268 | 0.9 |
| 11 | 316 | 0.95 | A₈B₂₅ | 364 | 16 |
| 12 | 372 | 17 | A₈B₂₆ | 229 | 11 |
| 13 | 296 | 1.1 | 16 | 404 | 0.019 |
| | | | 17 | 206 | 0.011 |

What is claimed is:

1. A compound of formula (I): 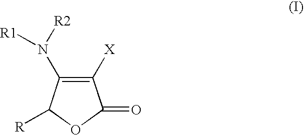
wherein X is selected from hydrogen, a halogen, a substituted or unsubstituted cyclic and heterocyclic moiety, substituted or unsubstituted, linear or branched alkyl, alkyloxy, alkylcarbonyl, alkyloxycarbonyl, alkenyl, alkenyloxy, alkenylcarbonyl, alkenyloxycarbonyl, alkynyl, alkynyloxy, alkynylcarbonyl, alkynyloxycarbonyl, aryl, benzyl, aryloxy, arlyoxy, arylcarbonyl, aryloxycarbonyl and sulphur equivalents of said oxy, carbonyl and oxycarbonyl moieties,
R is selected from hydrogen, a halogen, an amide, a substituted or unsubstituted cyclic and heterocyclic moiety, substituted or unsubstituted, linear or branched alkyl, alkyloxy, alkylcarbonyl, alkyloxycarbonyl, alkenyl, alkenyloxy, alkenylcarbonyl, alkenyloxycarbonyl, alkynyl, alkynyloxy, alkynylcarbonyl, alkynyloxycarbonyl, aryl, benzyl, araloxy arlyoxy, arylcarbonyl, aryloxycarbonyl and sulphur equivalents of said oxy, carbonyl and oxycarbonyl moieties, and
R¹ and R² are each independently selected from H, C₁₋₁₈ straight, branched or cyclic, saturated, unsaturated and aromatic hydrocarbyl groups, which aromatic groups may be heterocyclic, cyclic or acyclic and which may optionally be substituted by alkyl, alkoxy, or halo; or R¹ and R², when taken together with the N-atom to which they are bonded, may form an N-containing saturated, unsaturated or partially unsaturated ring system comprising 3 to 10 ring atoms selected from C, N and O, optionally substituted at any position of the ring by a substituent selected from a halogen, a substituted or unsubstituted cyclic and heterocyclic moiety, substituted or unsubstituted, linear or branched alkyl, alkyloxy, alkylcarbonyl, alkyloxycarbonyl, alkenyl, alkenyloxy, alkenylcarbonyl, alkenyloxycarbonyl, alkynyl, alkynyloxy, alkynylcarbonyl, alkynyloxycarbonyl, aryl, benzyl, aryloxy, arylcarbonyl, aryloxycarbonyl, sulphur equivalents of said oxy, carbonyl and oxycarbonyl moieties, and oxo.

2. A compound as claimed in claim 1, wherein said alkyl-containing moieties are C₁-C₁₈.

3. A compound as claimed in claim 1, wherein said alkenyl- and said alkynyl-containing moieties are C₂-C₁₈.

4. A compound as claimed in claim 1, wherein R¹ and R² taken together with the N-atom to which they are bonded, form an optionally-substituted: pyrrolidinyl, piperidinyl, benzimidazolyl, pyrrolyl, pyrazolyl, tetrahydropyrazinyl, dihydropyrazolyl, pyrazolyl, 2,3-dihydro-IH-indol-1-yl, pipetrazin-1-yl, morpholin-4-yl or pyrid-I-yl moiety.

5. A compound as claimed in claim 4, wherein substituents on the ring system formed by R¹ and R² are selected from C₁₋₆ alkyl or alkoxy, phenyl, benzyl, phenyl (C₂₋₄) alkenyl, phenoxy, benzyloxy, halo, oxo and alkyloxycarbonyl.

6. A compound as claimed in claim 4, wherein system formed by R₁ and R₂ is mono-or di-substituted.

7. A compound as claimed in claim 1, wherein R¹ and R² are, independently, H, C₁₋₆ alkyl, alkenyl, alkynyl, benzyl, and cyclohexyl.

8. A compound as claimed in claim 7, wherein one of R¹ and R² is H, C₁₋₆ alkyl or benzyl and the other is C₁₋₆ alkyl, phenyl, benzyl or phenyl(C₂₋₄) alkyl, cyclohexyl, 1,3-dihydro-3H-pyrazolyl or morpholin-4-yl.

9. A compound as claimed in claim 1, wherein X is selected from H, F, Br, Cl, I and methyl.

10. A compound as claimed in claim 1, wherein R is selected from H, halo, imidazolidinoyl, alkoxy, alkenoxy, alkynyloxy, alkylcarbonyloxy, alkylcarbonylmethyl, hydrazonoalkylmethyl optionally substituted phenyl, R³—N(R⁴CO)—, R³—N(R⁴)—CO—O—, and R³-N(R⁴)-O-, wherein R³ and R⁴ are independently selected from C₁₋₁₈ straight, branched or cyclic, saturated, unsaturated and aromatic hydrocarbyl groups, which aromatic groups may optionally be substituted by C₁₋₆ alkyl or alkoxy, and halo.

11. A compound as claimed in claim 10, wherein R³ is a C₆₋₁₀ aromatic group, selected from optionally substituted phenyl, naphthyl or benzyl.

12. A compound as claimed in claim 10, wherein R⁴ is H, methyl or ethyl.

13. A compound as claimed in claim 10, wherein R is selected from methoxy, ethenyloxy, propyn-2-yloxy, methylcarbonyloxy and optionally substituted phenyl.

14. A compound as claimed in claim 1 having the one of following formulae (II-IV)

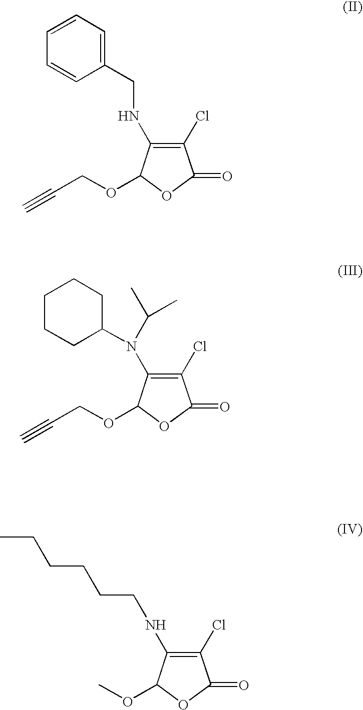

15. (canceled)

16. (canceled)

17. A method of treatment of a mammal afflicted with a CCK-receptor mediated condition, or prophylaxis in a mammal at risk of a CCK-receptor mediated condition comprising administering a therapeutically effective amount of a compound as claimed in claim 1.

18. (canceled)

19. The method of claim 17, wherein said CCK-receptor mediated condition is a GI disorder, a CNS disorder caused by CCK interaction with dopamine, another CNS disorder; oncologic disorder, disorder of appetite regulatory systems; Zollinger-Ellison syndrome; antral G cell hyperplasia; or pain.

20. The method of claim 19, wherein said GI disorder is selected from irritable bowel syndrome, gastro-oesophageal reflux disease or ulcers, excess pancreatic or gastric secretion, acute pancreitis, or motility disorders; said CNS disorder is selected from neuroleptic disorders, tardive dyskinesia, Parkinson's disease, psychosis or Gilles de la Tourette syndrome, said another CNS disorder is selected from anxiety disorders and panic disorders and said oncologic disorder is selected from small cell adenocarcinomas and primary tumours of the central nervous system glial and neuronal cells.

21. A method of inhibiting CCK receptor activity comprising contacting a composition comprising a CCK receptor with the compound of claim 1.

22. The method of claim 21, wherein said ligand is a selective CCK1 or CCK2 ligand.

23. A composition for the treatment or prophylaxis of a CCK-receptor mediated condition comprising the compound of claim 1 and a pharmaceutically acceptable carrier.
